(19) United States
(12) Patent Application Publication
(10) Pub. No.: US 2022/0275362 A1
(43) Pub. Date: Sep. 1, 2022

(54) METHODS FOR TAGGING AND ENCODING OF PRE-EXISTING COMPOUND LIBRARIES

(71) Applicant: X-Chem, Inc., Waltham, MA (US)

(72) Inventors: Anthony D. KEEFE, Cambridge, MA (US); Zhen CHEN, Boston, MA (US)

(73) Assignee: X-Chem, Inc., Waltham, MA (US)

(21) Appl. No.: 17/628,963

(22) PCT Filed: Jul. 24, 2020
(86) PCT No.: PCT/US2020/043419

§ 371 Date: Jan. 21, 2022

Related U.S. Application Data
(60) Provisional application No. 62/878,563, filed on Jul. 25, 2019.

(51) Int. Cl. C12N 15/10; C40B 50/04; C07K 1/13

(52) U.S. Cl. CPC C12N 15/1065; C40B 50/04; C07K 1/13; C12N 2310/531

(57) ABSTRACT

The present disclosure relates to methods of encoding pre-existing compounds with oligonucleotide tags. In particular, libraries of pre-existing compounds are tagged with oligonucleotides in order to encode identifying information, thereby improving methods of screening and identifying compounds having a desired property.

### SEQUENCE LISTING

The instant application contains a Sequence Listing which has been submitted electronically in ASCII format and is hereby incorporated by reference in its entirety. Said ASCII copy, created on Jul. 23, 2020, is named 50719-060WO2_Sequence_Listing_07.23.20_ST25 and is 3,747 bytes in size.

### BACKGROUND OF THE INVENTION

In general, this invention relates to DNA-encoded libraries of compounds and methods of using and creating such libraries. The invention also relates to compositions for use in such libraries.

Pre-existing compound libraries can provide a large number of diverse compounds and can be beneficial for drug discovery. Encoding such libraries with DNA tags could allow for rapid screening and interrogation of large numbers of pre-existing compounds against a large number of targets.

### SUMMARY OF THE INVENTION

The present invention features methods of tagging large libraries of pre-existing compounds with oligonucleotide tags that encode each member of the libraries with identifying information. The method optionally includes using orthogonal combinations of oligonucleotide tags in order to efficiently encode the pre-existing compounds. The pre-existing compounds, for example, are synthesized prior to the introduction of the encoding oligonucleotide tags. The oligonucleotide tags are covalently attached. Libraries of pre-existing compounds can be synthesized without the intentional introduction of a cross-linking group. The pre-existing compounds are encoded by conjugation to a bifunctional linker that is subsequently conjugated to a headpiece which is conjugated to oligonucleotides tags that encode the identity of the compound. When the tag combination identity is established, it may be used to determine the identity of the encoded molecule.

DNA-encoded chemical libraries including chemically synthesized small molecules that are created by the display of a single building block upon an encoding DNA sequence and its subsequent diversification with at least one additional chemical step and at least one additional conjugation to an additional encoding oligonucleotide. Such libraries contain combinatorial assemblages of chemically synthesized building block combinations encoded by corresponding combinatorial assemblages of encoding oligonucleotides. Determining the sequences of individual combinations of encoding oligonucleotides enables the determination of the chemical histories of the encoded chemical entities to which they are conjugated which therefore permits the determination of individual encoded chemical structures even when derived from a complex mixture. The utilization of such libraries in combination with affinity-mediated discovery processes is profoundly useful in the context of discovering combinatorically generated ligands to targets including therapeutically relevant targets such as disease-associated proteins.

However, not all chemical structures are readily accessed using chemical steps that are adaptable to combinatorial processes. For example, not all chemically synthesizable molecules are readily generated in a manner that is compatible with maintaining the enzymatic integrity of the encoding oligonucleotides. Additionally, many molecules of potential interest already exist in traditional (e.g., non-encoded) screening collections and their re-synthesis in a linkable form could be onerous, slow, and expensive.

This invention provides a means to begin with collections of pre-existing compounds and encode each member of the collections using combinations of encoding oligonucleotides in processes that encode large amounts of useful information. Such libraries of encoded molecules can then be screened against targets as a mixture. Screening linked versions of pre-existing libraries of compounds to find ligands to targets (e.g., therapeutic targets such as proteins) enables a robust method for discovering hit compounds (e.g., drug leads, drug candidates, and/or tool compounds).

In a first aspect, the invention features a method of producing an encoded chemical entity, the method including: (a) reacting a chemical entity with a bifunctional linker, the bifunctional linker including a carbene precursor group and a first cross-linking group, under conditions sufficient to produce a first conjugate including the chemical entity and the first cross-linking group; (b) reacting the first conjugate with a second conjugate, the second conjugate comprising an oligonucleotide headpiece and a second cross-linking group, under conditions sufficient to produce a third conjugate including the chemical entity and the oligonucleotide headpiece; and (c) ligating a first oligonucleotide tag to the oligonucleotide headpiece of the third conjugate, thereby producing an encoded chemical entity.

In some embodiments, the bifunctional linker is volatile.

In some embodiments, the bifunctional linker has the structure:

A-L¹-R¹ Formula I

where A is the carbene precursor group; L¹ is a linker; and R¹ is the first cross-linking group.

In some embodiments, the carbene precursor group is a photo-reactive carbene precursor group.

In some embodiments, the photo-reactive carbene precursor group is a diazirine.

In some embodiments, the carbene precursor group includes the structure:

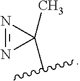 [SMILES: *C1(C)N=N1]

In some embodiments, L¹ is C₁-C₆ alkylene. In particular embodiments, L¹ is C₂ alkylene.

In some embodiments, the first cross-linking group is a sulfhydryl-reactive cross-linking group, an amino-reactive cross-linking group, a carboxyl-reactive cross-linking group, a carbonyl-reactive cross-linking group, or a triazole-forming cross-linking group.

In some embodiments, the first cross-linking group is a triazole-forming cross-linking group.

In some embodiments, the first cross-linking group is an azide.

In some embodiments, the bifunctional linker has the structure:

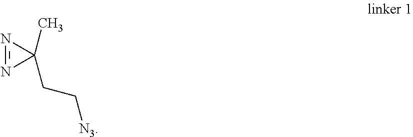

In some embodiments, the second conjugate has the structure:

B-L²-R² Formula II

where B is the oligonucleotide headpiece; L² is a linker; and R² is the second cross-linking group.

In some embodiments, the oligonucleotide headpiece comprises a hairpin structure.

In some embodiments, the second cross-linking group is a sulfhydryl-reactive cross-linking group, an amino-reactive cross-linking group, a carboxyl-reactive cross-linking group, a carbonyl-reactive cross-linking group, or a triazole-forming cross-linking group.

In some embodiments, the second cross-linking group is a triazole-forming cross-linking group.

In some embodiments, the second cross-linking group includes a dibenzocyclooctyne group.

In some embodiments, the second cross-linking group includes the structure:

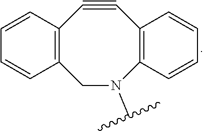 [SMILES: *N1Cc2ccccc2C#Cc2ccccc21]

In some embodiments, the method further comprises producing the second conjugate by reacting a fourth conjugate including an oligonucleotide headpiece and a cross-linking group with a fifth conjugate having the structure of Formula III:

R³-L³-R⁴ Formula III

where R³ and R⁴ are, independently, cross-linking groups; and L³ is a linker, under conditions sufficient to produce the second conjugate.

In some embodiments, R³ is a triazole-forming cross-linking group. In particular embodiments, R³ includes a dibenzocyclooctyne group. In still other embodiments, R³ includes the structure:

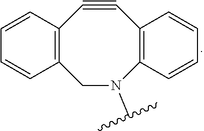 [SMILES: *N1Cc2ccccc2C#Cc2ccccc21]

In some embodiments, R⁴ is a sulfhydryl-reactive cross-linking group, an amino-reactive cross-linking group, a carboxyl-reactive cross-linking group, a carbonyl-reactive cross-linking group, or a triazole-forming cross-linking group. In particular embodiments, R⁴ is an amino-reactive cross-linking group. In still other embodiments, R⁴ includes a N-hydroxysuccinimide group.

In some embodiments, the second conjugate has the structure:

B-L⁴-R⁵ Formula IV

where B is the oligonucleotide headpiece; L⁴ is a linker; and R⁵ is the second cross-linking group.

In some embodiments, the reactive group is an amino group.

In some embodiments, the method further includes, prior to step (c), ligation of a headpiece extension sequence, e.g., a constant sequence to add a primer-binding sequence for PCR.

In some embodiments, the method further includes ligating one or more further tags to the encoded chemical entity after step (c).

In some embodiments, the method further includes ligating at least three further tags to the encoded chemical entity after step (c).

In some embodiments, the method comprises one-pot ligation. In some embodiments, the one-pot ligation includes the ligation of the headpiece extension sequence to the headpiece and the ligation of the at least three further tags to the encoded chemical entity.

In some embodiments, the first oligonucleotide tag and the one or more further tags comprise orthogonal overlap architectures.

In some embodiments, the method optionally includes ligating a tailpiece to the conjugate or encoded chemical entity. In some embodiments, the method further includes ligating a tailpiece to the conjugate or encoded chemical entity.

In some embodiments, the tailpiece includes one or more of a library-identifying sequence, a use sequence, or an origin sequence, as described herein.

In some embodiments, the chemical entity does not comprise an N—H or O—H bond.

In some embodiments, the conditions of step (b) do not comprise a metal catalyst.

In some embodiments, the method further comprises purifying the encoded chemical entity after step (c).

In some embodiments, the purifying comprises high performance liquid chromatography (HPLC).

In some embodiments, the conditions of step (a) comprises irradiation.

In another aspect, the invention features a library including a plurality of chemical entities produced by any of the foregoing methods.

In some embodiments, the plurality of chemical entities is not physically separated.

In some embodiments, the plurality of chemical entities includes at least 1,000,000 different compounds. In some embodiments, the plurality of chemical entities includes at least 5,000,000 different compounds. In some embodiments, the plurality of chemical entities includes at least 10,000,000 different compounds.

In some embodiments, the plurality of chemical entities includes about 500,000 to about 1,000,000 different compounds. In some embodiments, the plurality of chemical entities includes about 1,000,000 to about 5,000,000 different compounds. In some embodiments, the plurality of chemical entities includes about 1,000,000 to about 10,000,000 different compounds. In some embodiments, the plurality of chemical entities includes about 5,000,000 to about 10,000,000 different compounds. In some embodiments, the plurality of chemical entities includes about 5,000,000 to about 15,000,000 different compounds.

In yet another aspect, the invention features a method of screening a plurality of chemical entities, the method comprising: contacting a target with an encoded chemical entity prepared by any of the foregoing methods and/or any of the foregoing libraries; and selecting one or more encoded chemical entities having a predetermined characteristic for the target, as compared to a control, thereby screening a plurality of the chemical entities.

In some embodiments, the predetermined characteristic comprises increased binding for the target, as compared to a control.

In some embodiments, the method optionally includes ligating a tailpiece to the conjugate or encoded chemical entity. In some embodiments, the method further includes ligating a tailpiece to the conjugate or encoded chemical entity.

In some embodiments, the tailpiece includes one or more of a library-identifying sequence, a use sequence, or an origin sequence, as described herein.

### Definitions

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion, e.g., the interconversion illustrated in the scheme below:

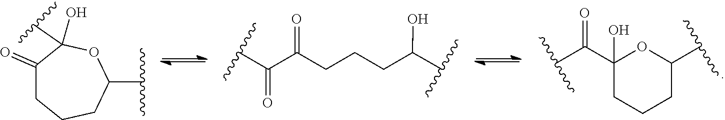 [SMILES: *C1CCCC(=O)C(*)(O)O1 ; *C(=O)C(=O)CCCC(*)O ; *C(=O)C1(O)CCCC(*)O1]

Those skilled in the art will appreciate that, in some embodiments, isotopes of compounds described herein may be prepared and/or utilized in accordance with the present invention. “Isotopes” refers to atoms having the same atomic number but different mass numbers resulting from a different number of neutrons in the nuclei. For example, isotopes of hydrogen include tritium and deuterium. In some embodiments, an isotopic substitution (e.g., substitution of hydrogen with deuterium) may alter the physicochemical properties of the molecules, such as metabolism and/or the rate of racemization of a chiral center.

As is known in the art, many chemical entities (in particular many organic molecules and/or many small molecules) can adopt a variety of different solid forms such as, for example, amorphous forms and/or crystalline forms (e.g., polymorphs, hydrates, and solvates). In some embodiments, such entities may be utilized in any form, including in any solid form. In some embodiments, such entities are utilized in a particular form, for example in a particular solid form.

In some embodiments, compounds described and/or depicted herein may be provided and/or utilized in salt form. In certain embodiments, compounds described and/or depicted herein may be provided and/or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl. Furthermore, where a compound includes a plurality of positions at which substitutes are disclosed in groups or in ranges, unless otherwise indicated, the present disclosure is intended to cover individual compounds and groups of compounds (e.g., genera and subgenera) containing each and every individual subcombination of members at each position.

Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g. alkyl) per se is optional.

By “about” is meant+/−10% of the recited value.

The term “alkyl,” as used herein, refers to saturated hydrocarbon groups containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by methyl, ethyl, n- and iso-propyl, n-, sec-, iso- and tert-butyl, neopentyl, and the like, and may be optionally substituted with one, two, three, or, in the case of alkyl groups of two carbons or more, four substituents independently selected from the group consisting of: (1) C₁₋₆ alkoxy; (2) C₁₋₆ alkylsulfinyl; (3) amino, as defined herein (e.g., unsubstituted amino (i.e., —NH₂) or a substituted amino (i.e., —N(R^{N1})₂, where R^{N1} is as defined for amino); (4) C₆₋₁₀ aryl-C₁₋₆ alkoxy; (5) azido; (6) halo; (7) (C₂₋₉ heterocyclyl)oxy; (8) hydroxyl, optionally substituted with an O-protecting group; (9) nitro; (10) oxo (e.g., carboxyaldehyde or acyl); (11) C₁₋₇ spirocyclyl; (12) thioalkoxy; (13) thiol; (14) —CO₂R^{A′}, optionally substituted with an O-protecting group and where R^{A′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alk-C₆₋₁₀ aryl, (f) amino-C₁₋₂₀alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (15) —C(O)NR^{B′}R^{C′}, where each of R^{B′} and R^{C′} is, independently, selected from the group consisting of (a) hydrogen, (b) C₁₋₆ alkyl, (c) C₆₋₁₀ aryl, and (d) C₁₋₆ alk-C₆₋₁₀ aryl; (16) —SO₂R^{D′}, where R^{D′} is selected from the group consisting of (a) C₁₋₆ alkyl, (b) C₆₋₁₀ aryl, (c) C₁₋₆ alk-C₆₋₁₀ aryl, and (d) hydroxyl; (17) —SO₂NR^{E′}R^{F′}, where each of R^{E′} and R^{F′} is, independently, selected from the group consisting of (a) hydrogen, (b) C₁₋₆ alkyl, (c) C₆₋₁₀ aryl and (d) C₁₋₆ alk-C₆₋₁₀ aryl; (18) —C(O)R^{G′}, where R^{G′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alk-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (19) —NR^{H′}C(O)R^{I′}, wherein R^{H′} is selected from the group consisting of (a1) hydrogen and (b1) C₁₋₆ alkyl, and R^{I′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (20) —NR^{J′}C(O)OR^{K′}, wherein R^{J′} is selected from the group consisting of (a1) hydrogen and (b1) C₁₋₆ alkyl, and R^{K′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (21) amidine; and (22) silyl groups such as trimethylsilyl, t-butyldimethylsilyl, and triisopropylsilyl. In some embodiments, each of these groups can be further substituted as described herein. For example, the alkylene group of a C₁-alkaryl can be further substituted with an oxo group to afford the respective aryloyl substituent.

The term “alkylene” and the prefix “alk-,” as used herein, represent a saturated divalent hydrocarbon group derived from a straight or branched chain saturated hydrocarbon by the removal of two hydrogen atoms, and is exemplified by methylene, ethylene, isopropylene, and the like. The term “C_{x-y} alkylene” and the prefix “C_{x-y} alk-” represent alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁₋₆, C₁₋₁₀, C₂₋₂₀, C₂₋₆, C₂₋₁₀, or C₂₋₂₀ alkylene). In some embodiments, the alkylene can be further substituted with 1, 2, 3, or 4 substituent groups as defined herein for an alkyl group.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, 2-butenyl, and the like. Alkenyls include both cis and trans isomers. Alkenyl groups may be optionally substituted with 1, 2, 3, or 4 substituent groups that are selected, independently, from amino, aryl, cycloalkyl, or heterocyclyl (e.g., heteroaryl), as defined herein, or any of the exemplary alkyl substituent groups described herein.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, 1-propynyl, and the like. Alkynyl groups may be optionally substituted with 1, 2, 3, or 4 substituent groups that are selected, independently, from aryl, cycloalkyl, or heterocyclyl (e.g., heteroaryl), as defined herein, or any of the exemplary alkyl substituent groups described herein.

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, alkaryl, cycloalkyl, alkcycloalkyl, carboxyalkyl (e.g., optionally substituted with an O-protecting group, such as optionally substituted arylalkoxycarbonyl groups or any described herein), sulfoalkyl, acyl (e.g., acetyl, trifluoroacetyl, or others described herein), alkoxycarbonylalkyl (e.g., optionally substituted with an O-protecting group, such as optionally substituted arylalkoxycarbonyl groups or any described herein), heterocyclyl (e.g., heteroaryl), or alkheterocyclyl (e.g., alkheteroaryl), wherein each of these recited R^{N1} groups can be optionally substituted, as defined herein for each group; or two R^{N1} combine to form a heterocyclyl or an N-protecting group, and wherein each R^{N2} is, independently, H, alkyl, or aryl. Amino groups can be an unsubstituted amino (i.e., —NH₂) or a substituted amino (i.e., —N(R^{N1})₂). In a preferred embodiment, amino is —NH₂ or —NHR^{N1}, wherein R^{N1} is, independently, OH, NO₂, NH₂, NR^{N2}₂, SO₂OR^{N2}, SO₂R^{N2}, SOR^{N2}, alkyl, carboxyalkyl, sulfoalkyl, acyl (e.g., acetyl, trifluoroacetyl, or others described herein), alkoxycarbonylalkyl (e.g., t-butoxycarbonylalkyl) or aryl, and each R^{N2} can be H, C₁₋₂₀ alkyl (e.g., C₁₋₆ alkyl), or C₆₋₁₀ aryl.

The term “amino acid,” as described herein, refers to a molecule having a side chain, an amino group, and an acid group (e.g., a carboxy group of —CO₂H or a sulfo group of —SO₃H), wherein the amino acid is attached to the parent molecular group by the side chain, amino group, or acid group (e.g., the side chain). As used herein, the term “amino acid” in its broadest sense, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, and/or substitution as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” is used to refer to a free amino acid; in some embodiments it is used to refer to an amino acid residue of a polypeptide. In some embodiments, the amino acid is attached to the parent molecular group by a carbonyl group, where the side chain or amino group is attached to the carbonyl group. In some embodiments, the amino acid is an α-amino acid. In certain embodiments, the amino acid is a 3-amino acid. In some embodiments, the amino acid is a γ-amino acid. Exemplary side chains include an optionally substituted alkyl, aryl, heterocyclyl, alkaryl, alkheterocyclyl, aminoalkyl, carbamoylalkyl, and carboxyalkyl. Exemplary amino acids include alanine, arginine, asparagine, aspartic acid, cysteine, glutamic acid, glutamine, glycine, histidine, hydroxynorvaline, isoleucine, leucine, lysine, methionine, norvaline, ornithine, phenylalanine, proline, pyrrolysine, selenocysteine, serine, taurine, threonine, tryptophan, tyrosine, and valine. Amino acid groups may be optionally substituted with one, two, three, or, in the case of amino acid groups of two carbons or more, four substituents independently selected from the group consisting of: (1) C₁₋₆ alkoxy; (2) C₁₋₆ alkylsulfinyl; (3) amino, as defined herein (e.g., unsubstituted amino (i.e., —NH₂) or a substituted amino (i.e., —N(R^{N1})₂, where R^{N1} is as defined for amino); (4) C₆₋₁₀ aryl-C₁₋₆ alkoxy; (5) azido; (6) halo; (7) (C₂₋₉ heterocyclyl)oxy; (8) hydroxyl; (9) nitro; (10) oxo (e.g., carboxyaldehyde or acyl); (11) C₁₋₇ spirocyclyl; (12) thioalkoxy; (13) thiol; (14) —CO₂R^{A′}, where R^{A′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alk-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (15) —C(O)NR^{B′}R^{C′}, where each of R^{B′} and R^{C′} is, independently, selected from the group consisting of (a) hydrogen, (b) C₁₋₆ alkyl, (c) C₆₋₁₀ aryl, and (d) C₁₋₆ alk-C₆₋₁₀ aryl; (16) —SO₂R^{D′}, where R^{D′} is selected from the group consisting of (a) C₁₋₆ alkyl, (b) C₆₋₁₀ aryl, (c) C₁₋₆ alk-C₆₋₁₀ aryl, and (d) hydroxyl; (17) —SO₂NR^{E′}R^{F′}, where each of R^{E′} and R^{F′} is, independently, selected from the group consisting of (a) hydrogen, (b) C₁₋₆ alkyl, (c) C₆₋₁₀ aryl and (d) C₁₋₆ alk-C₆₋₁₀ aryl; (18) —C(O)R^{G′}, where R^{G′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alk-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (19) —NR^{H′}C(O)R^{I′}, wherein R^{H′} is selected from the group consisting of (a1) hydrogen and (b1) C₁₋₆ alkyl, and R^{I′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (20) —NR^{J′}C(O)OR^{K′}, wherein R^{J′} is selected from the group consisting of (a1) hydrogen and (b1) C₁₋₆ alkyl, and R^{K′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; and (21) amidine. In some embodiments, each of these groups can be further substituted as described herein.

By “amino-reactive” or “amine-reactive” is meant a group which exhibits reactivity with amino groups (e.g., primary amino group, secondary amino group, or tertiary amino group). Exemplary, non-limiting amino-reactive groups include haloalkane, alkene (e.g., α,ρ-unsaturated carbonyl or vinylsulfone), epoxide, aldehyde, ketone, ester (e.g., N-hydroxysuccinimide (NHS) ester), carboxylic acid, isocyanate, sulfonyl chloride, acyl azide, anhydride, carbodiimide, carbonate, imidoester, pentafluorophenyl ester, and hydroxymethylphosphine.

The term “aryl,” as used herein, represents a mono-, bicyclic, or multicyclic carbocyclic ring system having one or two aromatic rings and is exemplified by phenyl, naphthyl, 1,2-dihydronaphthyl, 1,2,3,4-tetrahydronaphthyl, anthracenyl, phenanthrenyl, fluorenyl, indanyl, indenyl, and the like, and may be optionally substituted with 1, 2, 3, 4, or 5 substituents independently selected from the group consisting of: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alk-C₆₋₁₀ aryl; (8) azido; (9) C₃₋₈ cycloalkyl; (10) C₁₋₆ alk-C₃₋₈ cycloalkyl; (11) halo; (12) C₁₋₁₂ heterocyclyl (e.g., C₁₋₁₂ heteroaryl); (13) (C₁₋₁₂ heterocyclyl)oxy; (14) hydroxyl; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alk-C₆₋₁₀ aryl; (18) —(CH₂)_{q}CONR^{B′}R^{C′}, where q is an integer from zero to four and where R^{B′} and R^{C′} are independently selected from the group consisting of (a) hydrogen, (b) C₁₋₆ alkyl, (c) C₆₋₁₀ aryl, and (d) C₁₋₆ alk-C₆₋₁₀ aryl; (19) —(CH₂)_{q}SO₂R^{D′}, where q is an integer from zero to four and where R^{D′} is selected from the group consisting of (a) alkyl, (b) C₆₋₁₀ aryl, and (c) alk-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₁₋₆ alkyl, (c) C₆₋₁₀ aryl, and (d) C₁₋₆ alk-C₆₋₁₀ aryl; (21) thiol; (22) C₆₋₁₀ aryloxy; (23) C₃₋₈ cycloalkoxy; (24) C₆₋₁₀ aryl-C₁₋₆ alkoxy; (25) C₁₋₆ alk-C₁₋₁₂ heterocyclyl (e.g., C₁₋₆ alk-C₁₋₁₂ heteroaryl); (26) C₂₋₂₀ alkenyl; and (27) C₂₋₂₀ alkynyl. In some embodiments, each of these groups can be further substituted as described herein. For example, the alkylene group of a C₁-alkaryl or a C₁-alkheterocyclyl can be further substituted with an oxo group to afford the respective aryloyl and (heterocyclyl)oyl substituent group.

The “arylalkyl” group, which as used herein, represents an aryl group, as defined herein, attached to the parent molecular group through an alkylene group, as defined herein. Exemplary unsubstituted arylalkyl groups are from 7 to 30 carbons (e.g., from 7 to 16 or from 7 to 20 carbons, such as C₁₋₆ alk-C₆₋₁₀ aryl, C₁₋₁₀ alk-C₆₋₁₀ aryl, or C₁₋₂₀ alk-C₆₋₁₀ aryl). In some embodiments, the alkylene and the aryl each can be further substituted with 1, 2, 3, or 4 substituent groups as defined herein for the respective groups. Other groups preceded by the prefix “alk-” are defined in the same manner, where “alk” refers to a C₁₋₆ alkylene, unless otherwise noted, and the attached chemical structure is as defined herein.

The term “azido” represents an —N₃ group, which can also be represented as —N═N═N.

By “bifunctional” is meant having two reactive groups that allow for binding of two chemical moieties.

By “bifunctional linker,” as used herein, is meant a linker having two reactive groups (e.g., a carbene precursor group and a cross-linking group) that binds to (i) a chemical entity (e.g., pre-existing compound); and (ii) a conjugate including an oligonucleotide headpiece and a cross-linking group. Exemplary bifunctional linkers are provided herein.

By “binding” is meant attaching by a covalent bond or a non-covalent bond. Non-covalent bonds include those formed by van der Waals forces, hydrogen bonds, ionic bonds, entrapment or physical encapsulation, absorption, adsorption, and/or other intermolecular forces. Binding can be effectuated by any useful means, such as by enzymatic binding (e.g., enzymatic ligation to provide an enzymatic linkage) or by chemical binding (e.g., chemical ligation to provide a chemical linkage).

By “carbene” is meant a neutral carbon atom with a valence of two and two unshared valence electrons. A general formula for a structure including a carbene group is as follows:

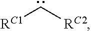

where each of R^{C1} and R^{C2} is H, optionally substituted C₁-C₁₂ alkyl (e.g., unsubstituted C₁-C₁₂ alkyl or C₁-C₁₂ alkyl substituted with one or more of halo, oxo, C₁-C₁₂ alkyl, C₁-C₁₂ heteroalkyl, C₃-C₁₀ carbocyclyl, C₆-C₁₀ aryl, C₂-C₉ heterocyclyl, or C₂-C₉ heteroaryl), or optionally substituted C₁-C₁₂ heteroalkyl (e.g., unsubstituted C₁-C₁₂ heteroalkyl or C₁-C₁₂ heteroalkyl substituted with one or more of halo, oxo, C₁-C₁₂ alkyl, C₁-C₁₂ heteroalkyl, C₃-C₁₀ carbocyclyl, C₆-C₁₀ aryl, C₂-C₉ heterocyclyl, or C₂-C₉ heteroaryl).

By “carbene precursor group” is meant a functional group that undergoes chemical reaction to generate a carbene group. Carbene precursor groups are known in the art, e.g., diazirines.

The terms “carbocyclic” and “carbocyclyl,” as used herein, refer to an optionally substituted C₃₋₁₂ monocyclic, bicyclic, or tricyclic non-aromatic ring structure in which the rings are formed by carbon atoms. Carbocyclic structures include cycloalkyl, cycloalkenyl, and cycloalkynyl groups. The “carbocyclylalkyl” group, which as used herein, represents a carbocyclic group, as defined herein, attached to the parent molecular group through an alkylene group, as defined herein. Exemplary unsubstituted carbocyclylalkyl groups are from 7 to 30 carbons (e.g., from 7 to 16 or from 7 to 20 carbons, such as C₁₋₆ alk-C₆₋₁₀ carbocyclyl, C₁₋₁₀ alk-C₆₋₁₀ carbocyclyl, or C₁₋₂₀ alk-C₆₋₁₀ carbocyclyl). In some embodiments, the alkylene and the carbocyclyl each can be further substituted with 1, 2, 3, or 4 substituent groups as defined herein for the respective groups. Other groups preceded by the prefix “alk-” are defined in the same manner, where “alk” refers to a C₁₋₆ alkylene, unless otherwise noted, and the attached chemical structure is as defined herein.

The term “carbonyl,” as used herein, represents a C(O) group, which can also be represented as C═O.

By “carbonyl-reactive” is meant a group which exhibits reactivity with carbonyl groups, i.e., groups containing —C(O)— (e.g., aldehyde, ketone, and acyl halide). Exemplary, non-limiting carbonyl-reactive groups include hydrazide, amine (e.g., alkoxyamine), and hydroxyl.

By “carboxyl-reactive” is meant a group which exhibits reactivity with carboxyl groups, i.e., —COOH. Exemplary, non-limiting carboxyl-reactive groups include carbodiimide, amine, and hydroxyl.

The term “carboxy,” as used herein, means —CO₂H.

By “chemical entity” is meant a compound comprising one or more building blocks and optionally one or more scaffolds. The chemical entity can be any small molecule or peptide drug or drug candidate designed or built to have one or more desired characteristics, e.g., capacity to bind a biological target, solubility, availability of hydrogen bond donors and acceptors, rotational degrees of freedom of the bonds, positive charge, negative charge, and the like. In certain embodiments, the chemical entity can be reacted further as a bifunctional or trifunctional (or greater) entity.

By “chemical-reactive group” is meant a reactive group that participates in a modular reaction, thus producing a linkage. Exemplary reactions and reactive groups include those selected from a Huisgen 1,3-dipolar cycloaddition reaction with a triazole-forming pair of an optionally substituted alkynyl group and an optionally substituted azido group; a Diels-Alder reaction with a pair of an optionally substituted diene having a 4 π-electron system and an optionally substituted dienophile or an optionally substituted heterodienophile having a 2 π-electron system; a ring opening reaction with a nucleophile and a strained heterocyclyl electrophile; a splint ligation reaction with a phosphorothioate group and an iodo group; and a reductive amination reaction with an aldehyde group and an amino group, as described herein.

By “complementary” is meant a sequence capable of hybridizing, as defined herein, to form secondary structure (a duplex or a double-stranded portion of a nucleic acid molecule). The complementarity need not be perfect but may include one or more mismatches at one, two, three, or more nucleotides. For example, complementary sequence may contain nucleobases that can form hydrogen bonds according to Watson-Crick base-pairing rules (e.g., G with C, A with T or A with U) or other hydrogen bonding motifs (e.g., diaminopurine with T, 5-methyl C with G, 2-thiothymidine with A, inosine with C, pseudoisocytosine with G). The sequence and its complementary sequence can be present in the same oligonucleotide or in different oligonucleotides.

By “connector” of an oligonucleotide tag is meant a portion of the tag at or in proximity to the 5′- or 3′-terminus having a fixed sequence. A 5′-connector is located at or in proximity to the 5′-terminus of an oligonucleotide, and a 3′-connector is located at or in proximity to the 3′-terminus of an oligonucleotide. When present in a conjugate or encoded chemical entity, each 5′-connector may be the same or different, and each 3′-connector may be the same or different. In an exemplary, non-limiting conjugate or encoded chemical entity having more than one tags, each tag can include a 5′-connector and a 3′-connector, where each 5′-connector has the same sequence and each 3′-connector has the same sequence (e.g., where the sequence of the 5′-connector can be the same or different from the sequence of the 3′-connector). In another exemplary, non-limiting conjugate or encoded chemical entity, the sequence of the 5′-connector is designed to be complementary, as defined herein, to the sequence of the 3′-connector (e.g., to allow for hybridization between 5′- and 3′-connectors). The connector can optionally include one or more groups allowing for a linkage (e.g., a linkage for which a polymerase has reduced ability to read or translocate through, such as a chemical linkage).

By “constant” or “fixed constant” sequence is meant a sequence of an oligonucleotide that does not encode information. Non-limiting, exemplary portions of a conjugate or encoded chemical entity having a constant sequence include a primer-binding region, a 5′-connector, or a 3′-connector. The headpiece can encode information (thus, a tag) or alternatively not encode information (thus, a constant sequence). Similarly, the tailpiece can encode or not encode information.

As used herein, the term “cross-linking group” refers to a group comprising a reactive functional group capable of chemically attaching to specific functional groups (e.g., primary amines, sulfhydryls) on proteins or other molecules. A “moiety capable of a chemoselective reaction with an amino acid,” as used herein refers to a moiety comprising a reactive functional group capable of chemically attaching to a functional group of a natural or non-natural amino acid (e.g., primary and secondary amines, sulfhydryls, alcohols, carboxyl groups, carbonyls, or triazole forming functional groups such as azides or alkynes). Examples of cross-linking groups include sulfhydryl-reactive cross-linking groups (e.g., groups comprising maleimides, haloacetyls, pyridyldisulfides, thiosulfonates, or vinylsulfones), amine-reactive cross-linking groups (e.g., groups comprising esters such as NHS esters, imidoesters, and pentafluorophenyl esters, or hydroxymethylphosphine), carboxyl-reactive cross-linking groups (e.g., groups comprising primary or secondary amines, alcohols, or thiols), carbonyl-reactive cross-linking groups (e.g., groups comprising hydrazides or alkoxyamines), and triazole-forming cross-linking groups (e.g., groups comprising azides or alkynes).

The term “cyano,” as used herein, represents an —CN group.

The term “cycloalkyl,” as used herein represents a monovalent saturated or unsaturated non-aromatic cyclic hydrocarbon group from three to eight carbons, unless otherwise specified, and is exemplified by cyclopropyl, cyclobutyl, cyclopentyl, cyclohexyl, cycloheptyl, bicycle heptyl, and the like. When the cycloalkyl group includes one carbon-carbon double bond, the cycloalkyl group can be referred to as a “cycloalkenyl” group. Exemplary cycloalkenyl groups include cyclopentenyl, cyclohexenyl, and the like. The cycloalkyl groups of this invention can be optionally substituted with: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alk-C₆₋₁₀ aryl; (8) azido; (9) C₃₋₈ cycloalkyl; (10) C₁₋₆ alk-C₃₋₈ cycloalkyl; (11) halo; (12) C₁₋₁₂ heterocyclyl (e.g., C₁₋₁₂ heteroaryl); (13) (C₁₋₁₂ heterocyclyl)oxy; (14) hydroxyl; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alk-C₆₋₁₀ aryl; (18) —(CH₂)_{q}CONR^{B′}R^{C′}, where q is an integer from zero to four and where R^{B′} and R^{C′} are independently selected from the group consisting of (a) hydrogen, (b) C₆₋₁₀ alkyl, (c) C₆₋₁₀ aryl, and (d) C₁₋₆ alk-C₆₋₁₀ aryl; (19) —(CH₂)_{q}SO₂R^{D′}, where q is an integer from zero to four and where R^{D′} is selected from the group consisting of (a) C₆₋₁₀ alkyl, (b) C₆₋₁₀ aryl, and (c) C₁₋₆ alk-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₆₋₁₀ alkyl, (c) C₆₋₁₀ aryl, and (d) C₁₋₆ alk-C₆₋₁₀ aryl; (21) thiol; (22) C₆₋₁₀ aryloxy; (23) C₃₋₈ cycloalkoxy; (24) C₆₋₁₀ aryl-C₁₋₆ alkoxy; (25) C₁₋₆ alk-C₁₋₁₂ heterocyclyl (e.g., C₁₋₆ alk-C₁₋₁₂ heteroaryl); (26) oxo; (27) C₂₋₂₀ alkenyl; and (28) C₂₋₂₀ alkynyl. In some embodiments, each of these groups can be further substituted as described herein. For example, the alkylene group of a C₁-alkaryl or a C₁-alkheterocyclyl can be further substituted with an oxo group to afford the respective aryloyl and (heterocyclyl)oyl substituent group.

The “cycloalkylalkyl” group, which as used herein, represents a cycloalkyl group, as defined herein, attached to the parent molecular group through an alkylene group, as defined herein (e.g., an alkylene group of from 1 to 4, from 1 to 6, from 1 to 10, or form 1 to 20 carbons). In some embodiments, the alkylene and the cycloalkyl each can be further substituted with 1, 2, 3, or 4 substituent groups as defined herein for the respective group.

The term “diastereomer,” as used herein means stereoisomers that are not mirror images of one another and are non-superimposable on one another.

The term “enantiomer,” as used herein, means each individual optically active form of a compound, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “halo,” as used herein, represents a halogen selected from bromine, chlorine, iodine, or fluorine.

By “hairpin structure” is meant a structure formed when two regions of a single-stranded oligonucleotide, usually complementary in nucleotide sequence when read in opposite directions, base-pair to form a double helix that ends in an unpaired loop.

By “headpiece” is meant a chemical structure for library synthesis that is operatively linked to a component of a chemical entity and to a tag, e.g., a starting oligonucleotide. Optionally, a headpiece may contain few or no nucleotides but may provide a point at which they may be operatively associated.

Optionally, a bifunctional linker connects the headpiece to the component.

The term “heteroalkyl,” as used herein, refers to an alkyl group, as defined herein, in which one or two of the constituent carbon atoms have each been replaced by nitrogen, oxygen, or sulfur. In some embodiments, the heteroalkyl group can be further substituted with 1, 2, 3, or 4 substituent groups as described herein for alkyl groups. The terms “heteroalkenyl” and heteroalkynyl,” as used herein refer to alkenyl and alkynyl groups, as defined herein, respectively, in which one or two of the constituent carbon atoms have each been replaced by nitrogen, oxygen, or sulfur. In some embodiments, the heteroalkenyl and heteroalkynyl groups can be further substituted with 1, 2, 3, or 4 substituent groups as described herein for alkyl groups.

The term “heteroaryl,” as used herein, represents that subset of heterocyclyls, as defined herein, which are aromatic: i.e., they contain 4n+2 pi electrons within the mono- or multicyclic ring system. Exemplary unsubstituted heteroaryl groups are of 1 to 12 (e.g., 1 to 11, 1 to 10, 1 to 9, 2 to 12, 2 to 11, 2 to 10, or 2 to 9) carbons. In some embodiment, the heteroaryl is substituted with 1, 2, 3, or 4 substituents groups as defined for a heterocyclyl group.

The term “heteroarylalkyl” refers to a heteroaryl group, as defined herein, attached to the parent molecular group through an alkylene group, as defined herein. Exemplary unsubstituted heteroarylalkyl groups are from 2 to 32 carbons (e.g., from 2 to 22, from 2 to 18, from 2 to 17, from 2 to 16, from 3 to 15, from 2 to 14, from 2 to 13, or from 2 to 12 carbons, such as C₁₋₆ alk-C₁₋₁₂ heteroaryl, C₁₋₁₀ alk-C₁₋₁₂ heteroaryl, or C₁₋₂₀ alk-C₁₋₁₂ heteroaryl). In some embodiments, the alkylene and the heteroaryl each can be further substituted with 1, 2, 3, or 4 substituent groups as defined herein for the respective group. Heteroarylalkyl groups are a subset of heterocyclylalkyl groups.

The term “heterocyclyl,” as used herein represents a 5-, 6- or 7-membered ring, unless otherwise specified, containing one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocyclyl groups are of 1 to 12 (e.g., 1 to 11, 1 to 10, 1 to 9, 2 to 12, 2 to 11, 2 to 10, or 2 to 9) carbons. The term “heterocyclyl” also represents a heterocyclic compound having a bridged multicyclic structure in which one or more carbons and/or heteroatoms bridges two non-adjacent members of a monocyclic ring, e.g., a quinuclidinyl group. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring, such as indolyl, quinolyl, isoquinolyl, tetrahydroquinolyl, benzofuryl, benzothienyl and the like. Examples of fused heterocyclyls include tropanes and 1,2,3,5,8,8a-hexahydroindolizine. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidinyl, morpholinyl, thiomorpholinyl, thiazolyl, thiazolidinyl, isothiazolyl, isothiazolidinyl, indolyl, indazolyl, quinolyl, isoquinolyl, quinoxalinyl, dihydroquinoxalinyl, quinazolinyl, cinnolinyl, phthalazinyl, benzimidazolyl, benzothiazolyl, benzoxazolyl, benzothiadiazolyl, furyl, thienyl, thiazolidinyl, isothiazolyl, triazolyl, tetrazolyl, oxadiazolyl (e.g., 1,2,3-oxadiazolyl), purinyl, thiadiazolyl (e.g., 1,2,3-thiadiazolyl), tetrahydrofuranyl, dihydrofuranyl, tetrahydrothienyl, dihydrothienyl, dihydroindolyl, dihydroquinolyl, tetrahydroquinolyl, tetrahydroisoquinolyl, dihydroisoquinolyl, pyranyl, dihydropyranyl, dithiazolyl, benzofuranyl, isobenzofuranyl, benzothienyl, and the like, including dihydro and tetrahydro forms thereof, where one or more double bonds are reduced and replaced with hydrogens. Still other exemplary heterocyclyls include: 2,3,4,5-tetrahydro-2-oxo-oxazolyl; 2,3-dihydro-2-oxo-1H-imidazolyl; 2,3,4,5-tetrahydro-5-oxo-1H-pyrazolyl (e.g., 2,3,4,5-tetrahydro-2-phenyl-5-oxo-1H-pyrazolyl); 2,3,4,5-tetrahydro-2,4-dioxo-1H-imidazolyl (e.g., 2,3,4,5-tetrahydro-2,4-dioxo-5-methyl-5-phenyl-1H-imidazolyl); 2,3-dihydro-2-thioxo-1,3,4-oxadiazolyl (e.g., 2,3-dihydro-2-thioxo-5-phenyl-1,3,4-oxadiazolyl); 4,5-dihydro-5-oxo-1H-triazolyl (e.g., 4,5-dihydro-3-methyl-4-amino 5-oxo-1H-triazolyl); 1,2,3,4-tetrahydro-2,4-dioxopyridinyl (e.g., 1,2,3,4-tetrahydro-2,4-dioxo-3,3-diethylpyridinyl); 2,6-dioxo-piperidinyl (e.g., 2,6-dioxo-3-ethyl-3-phenylpiperidinyl); 1,6-dihydro-6-oxopyridiminyl; 1,6-dihydro-4-oxopyrimidinyl (e.g., 2-(methylthio)-1,6-dihydro-4-oxo-5-methylpyrimidin-1-yl); 1,2,3,4-tetrahydro-2,4-dioxopyrimidinyl (e.g., 1,2,3,4-tetrahydro-2,4-dioxo-3-ethylpyrimidinyl); 1,6-dihydro-6-oxo-pyridazinyl (e.g., 1,6-dihydro-6-oxo-3-ethylpyridazinyl); 1,6-dihydro-6-oxo-1,2,4-triazinyl (e.g., 1,6-dihydro-5-isopropyl-6-oxo-1,2,4-triazinyl); 2,3-dihydro-2-oxo-1H-indolyl (e.g., 3,3-dimethyl-2,3-dihydro-2-oxo-1H-indolyl and 2,3-dihydro-2-oxo-3,3′-spiropropane-1H-indol-1-yl); 1,3-dihydro-1-oxo-2H-iso-indolyl; 1,3-dihydro-1,3-dioxo-2H-iso-indolyl; 1H-benzopyrazolyl (e.g., 1-(ethoxycarbonyl)-1H-benzopyrazolyl); 2,3-dihydro-2-oxo-1H-benzimidazolyl (e.g., 3-ethyl-2,3-dihydro-2-oxo-1H-benzimidazolyl); 2,3-dihydro-2-oxo-benzoxazolyl (e.g., 5-chloro-2,3-dihydro-2-oxo-benzoxazolyl); 2,3-dihydro-2-oxo-benzoxazolyl; 2-oxo-2H-benzopyranyl; 1,4-benzodioxanyl; 1,3-benzodioxanyl; 2,3-dihydro-3-oxo,4H-1,3-benzothiazinyl; 3,4-dihydro-4-oxo-3H-quinazolinyl (e.g., 2-methyl-3,4-dihydro-4-oxo-3H-quinazolinyl); 1,2,3,4-tetrahydro-2,4-dioxo-3H-quinazolyl (e.g., 1-ethyl-1,2,3,4-tetrahydro-2,4-dioxo-3H-quinazolyl); 1,2,3,6-tetrahydro-2,6-dioxo-7H-purinyl (e.g., 1,2,3,6-tetrahydro-1,3-dimethyl-2,6-dioxo-7H-purinyl); 1,2,3,6-tetrahydro-2,6-dioxo-1H-purinyl (e.g., 1,2,3,6-tetrahydro-3,7-dimethyl-2,6-dioxo-1H-purinyl); 2-oxobenzo[c,d]indolyl; 1,1-dioxo-2H-naphth[1,8-c,d]isothiazolyl; and 1,8-naphthylenedicarboxamido. Additional heterocyclics include 3,3a,4,5,6,6a-hexahydro-pyrrolo[3,4-b]pyrrol-(2H)-yl, and 2,5-diazabicyclo[2.2.1]heptan-2-yl, homopiperazinyl (or diazepanyl), tetrahydropyranyl, dithiazolyl, benzofuranyl, benzothienyl, oxepanyl, thiepanyl, azocanyl, oxecanyl, and thiocanyl. Heterocyclic groups also include groups of the formula

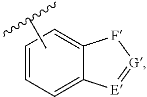

where E′ is selected from the group consisting of —N— and —CH—; F′ is selected from the group consisting of —N═CH—, —NH—CH₂—, —NH—C(O)—, —NH—, —CH═N—, —CH₂—NH—, —C(O)—NH—, —CH═CH—, —CH₂—, —CH₂CH₂—, —CH₂O—, —OCH₂—, —O—, and —S—; and G′ is selected from the group consisting of —CH— and —N—. Any of the heterocyclyl groups mentioned herein may be optionally substituted with one, two, three, four or five substituents independently selected from the group consisting of: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alk-C₆₋₁₀ aryl; (8) azido; (9) C₃₋₈ cycloalkyl; (10) C₁₋₆ alk-C₃₋₈ cycloalkyl; (11) halo; (12) C₁₋₁₂ heterocyclyl (e.g., C₂₋₁₂ heteroaryl); (13) (C₁₋₁₂ heterocyclyl)oxy; (14) hydroxyl; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alk-C₆₋₁₀ aryl; (18) —(CH₂)_{q}CONR^{B′}R^{C′}, where q is an integer from zero to four and where R^{B′} and R^{C′} are independently selected from the group consisting of (a) hydrogen, (b) C₁₋₆ alkyl, (c) C₆₋₁₀ aryl, and (d) C₁₋₆ alk-C₆₋₁₀ aryl; (19) —(CH₂)_{q}SO₂R^{D′}, where q is an integer from zero to four and where R^{D′} is selected from the group consisting of (a) C₁₋₆ alkyl, (b) C₆₋₁₀ aryl, and (c) C₁₋₆ alk-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₁₋₆ alkyl, (c) C₆₋₁₀ aryl, and (d) C₁₋₆ alk-C₆₋₁₀ aryl; (21) thiol; (22) C₆₋₁₀ aryloxy; (23) C₃₋₈ cycloalkoxy; (24) arylalkoxy; (25) C₁₋₆ alk-C₁₋₁₂ heterocyclyl (e.g., C₁₋₆ alk-C₁₋₁₂ heteroaryl); (26) oxo; (27) (C₁₋₁₂ heterocyclyl)imino; (28) C₂₋₂₀ alkenyl; and (29) C₂₋₂₀ alkynyl. In some embodiments, each of these groups can be further substituted as described herein. For example, the alkylene group of a C₁-alkaryl or a C₁-alkheterocyclyl can be further substituted with an oxo group to afford the respective aryloyl and (heterocyclyl)oyl substituent group.

The “heterocyclylalkyl” group, which as used herein, represents a heterocyclyl group, as defined herein, attached to the parent molecular group through an alkylene group, as defined herein. Exemplary unsubstituted heterocyclylalkyl groups are from 2 to 32 carbons (e.g., from 2 to 22, from 2 to 18, from 2 to 17, from 2 to 16, from 3 to 15, from 2 to 14, from 2 to 13, or from 2 to 12 carbons, such as C₁₋₆ alk-C₁₋₁₂ heterocyclyl, C₁₋₁₀ alk-C₁₋₁₂ heterocyclyl, or C₁₋₂₀ alk-C₁₋₁₂ heterocyclyl). In some embodiments, the alkylene and the heterocyclyl each can be further substituted with 1, 2, 3, or 4 substituent groups as defined herein for the respective group.

By “hybridize” is meant to pair to form a double-stranded molecule between complementary oligonucleotides, or portions thereof, under various conditions of stringency. (See, e.g., Wahl, G. M. and S. L. Berger (1987) Methods Enzymol. 152:399; Kimmel, A. R. (1987) Methods Enzymol. 152:507.) For example, high stringency hybridization can be obtained with a salt concentration ordinarily less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide or at least about 50% formamide. High stringency hybridization temperature conditions will ordinarily include temperatures of at least about 30° C., 37° C., or 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In an alternative embodiment, hybridization will occur at 37° C. in 500 mM NaCl, 50 mM trisodium citrate, 1% SDS, 35% formamide, and 100 μg/ml denatured salmon sperm DNA (ssDNA). In a further alternative embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, high stringency salt concentrations for the wash steps may be, e.g., less than about 30 mM NaCl and 3 mM trisodium citrate, or less than about 15 mM NaCl and 1.5 mM trisodium citrate. High stringency temperature conditions for the wash steps will ordinarily include a temperature of, e.g., at least about 25° C., 42° C., or 68° C. In one embodiment, wash steps will occur at 25° C. in 30 mM NaCl, 3 mM trisodium citrate, and 0.1% SDS. In an alternative embodiment, wash steps will occur at 42° C. in 15 mM NaCl, 1.5 mM trisodium citrate, and 0.1% SDS. In a further alternative embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

The term “hydrocarbon,” as used herein, represents a group consisting only of carbon and hydrogen atoms.

The term “hydroxyl,” as used herein, represents an —OH group. In some embodiments, the hydroxyl group can be substituted with 1, 2, 3, or 4 substituent groups (e.g., O-protecting groups) as defined herein for an alkyl.

The term “isomer,” as used herein, means any tautomer, stereoisomer, enantiomer, or diastereomer of any compound. It is recognized that the compounds can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

By “library” is meant a collection of molecules or chemical entities. Optionally, the molecules or chemical entities are bound to one or more oligonucleotides that encodes for the molecules or portions of the chemical entity. A library includes at least two members and may include at least 1,000 members, at least 10,000 members, at least 100,000 members, at least 1,000,000 members, at least 5,000,000 members, at least 10,000,000 members, at least 100,000,000 members, at least 1,000,000,000 members, at least 10,000,000,000 members, or at least 100,000,000,000 members.

By “linkage” is meant a chemical connecting entity that allows for operatively associating two or more chemical structures, for example, where the linkage is present between the headpiece and one or more tags, between two tags, or between a tag and a tailpiece. The chemical connecting entity can be a non-covalent bond (e.g., as described herein), a covalent bond, or a reaction product between two functional groups. By “chemical linkage” is meant a linkage formed by a non-enzymatic, chemical reaction between two functional groups. Exemplary, non-limiting functional groups include a chemical-reactive group, a photo-reactive group, an intercalating moiety, or a cross-linking oligonucleotide (e.g., as described herein). By “enzymatic linkage” is meant an internucleotide or internucleoside linkage formed by an enzyme. Exemplary, non-limiting enzymes include a kinase, a polymerase, a ligase, or combinations thereof. By a linkage “for which a polymerase has reduced ability to read or translocate through” is meant a linkage, when present in an oligonucleotide template, that provides a reduced amount of elongated and/or amplified products by a polymerase, as compared to a control oligonucleotide lacking the linkage. Exemplary, non-limiting methods for determining such a linkage include primer extension as assessed by PCR analysis (e.g., quantitative PCR), RT-PCR analysis, liquid chromatography-mass spectrometry, sequence demographics, or other methods. Exemplary, non-limiting polymerases include DNA polymerases and RNA polymerases, such as DNA polymerase I, DNA polymerase II, DNA polymerase III, DNA polymerase VI, Taq DNA polymerase, Deep VentR™ DNA Polymerase (high-fidelity thermophilic DNA polymerase, available from New England Biolabs), T7 DNA polymerase, T4 DNA polymerase, RNA polymerase I, RNA polymerase II, RNA polymerase III, or T7 RNA polymerase.

The term “N-protected amino,” as used herein, refers to an amino group, as defined herein, to which is attached one or two N-protecting groups, as defined herein.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999), which is incorporated herein by reference. N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, alkaryl groups such as benzyl, triphenylmethyl, benzyloxymethyl, and the like and silyl groups, such as trimethylsilyl, and the like. Preferred N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

By “oligonucleotide” is meant a polymer of nucleotides having a 5′-terminus, a 3′-terminus, and one or more nucleotides at the internal position between the 5′- and 3-termini. The oligonucleotide may include DNA, RNA, or any derivative thereof known in the art that can be synthesized and used for base-pair recognition. The oligonucleotide does not have to have contiguous bases but can be interspersed with linker moieties. The oligonucleotide polymer and nucleotide (e.g., modified DNA or RNA) may include natural bases (e.g., adenosine, thymidine, guanosine, cytidine, uridine, deoxyadenosine, deoxythymidine, deoxyguanosine, deoxycytidine, inosine, or diamino purine), base analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), modified bases (e.g., 2′-substituted nucleotides, such as 2′-O-methylated bases and 2′-fluoro bases), intercalated bases, modified sugars (e.g., 2′-fluororibose; ribose; 2′-deoxyribose; arabinose; hexose; anhydrohexitol; altritol; mannitol; cyclohexanyl; cyclohexenyl; morpholino that also has a phosphoramidate backbone; locked nucleic acids (LNA, e.g., where the 2′-hydroxyl of the ribose is connected by a C₁₋₆ alkylene or C₁₋₆ heteroalkylene bridge to the 4′-carbon of the same ribose sugar, where exemplary bridges included methylene, propylene, ether, or amino bridges); glycol nucleic acid (GNA, e.g., R-GNA or S-GNA, where ribose is replaced by glycol units attached to phosphodiester bonds); threose nucleic acid (TNA, where ribose is replace with α-L-threofuranosyl-(3′→2′)); and/or replacement of the oxygen in ribose (e.g., with S, Se, or alkylene, such as methylene or ethylene)), modified backbones (e.g., peptide nucleic acid (PNA), where 2-amino-ethyl-glycine linkages replace the ribose and phosphodiester backbone), and/or modified phosphate groups (e.g., phosphorothioates, 5′-N-phosphoramidites, phosphoroselenates, boranophosphates, boranophosphate esters, hydrogen phosphonates, phosphoramidates, phosphorodiamidates, alkyl or aryl phosphonates, phosphotriesters, bridged phosphoramidates, bridged phosphorothioates, and bridged methylene-phosphonates). The oligonucleotide can be single-stranded (e.g., hairpin), double-stranded, or possess other secondary or tertiary structures (e.g., stem-loop structures, double helixes, triplexes, quadruplexes, etc.).

By “one-pot ligation” is meant a ligation method in which at least two successive ligations (e.g., two ligations, three ligations, four ligations, five ligations, six ligations, seven ligations, eight ligations, nine ligations, ten ligations, or more than ten ligations) are conducted together in one reactor or one reaction vessel. Typically, a one-pot ligation avoids separation process steps and purification of intermediates.

By “operatively linked” or “operatively associated” is meant that two or more chemical structures are directly or indirectly linked together in such a way as to remain linked through the various manipulations they are expected to undergo. Typically, the chemical entity and the headpiece are operatively associated in an indirect manner (e.g., covalently via an appropriate linker). For example, the linker may be a bifunctional moiety with a site of attachment for chemical entity and a site of attachment for the headpiece.

The term “O-protecting group,” as used herein, represents those groups intended to protect an oxygen containing (e.g., phenol, hydroxyl, or carbonyl) group against undesirable reactions during synthetic procedures. Commonly used O-protecting groups are disclosed in Greene, “Protective Groups in Organic Synthesis,” 3^{rd} Edition (John Wiley & Sons, New York, 1999), which is incorporated herein by reference. Exemplary O-protecting groups include acyl, aryloyl, or carbamyl groups, such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl; alkylcarbonyl groups, such as acyl, acetyl, propionyl, pivaloyl, and the like; optionally substituted arylcarbonyl groups, such as benzoyl; silyl groups, such as trimethylsilyl (TMS), tert-butyldimethylsilyl (TBDMS), tri-iso-propylsilyloxymethyl (TOM), triisopropylsilyl (TIPS), and the like; ether-forming groups with the hydroxyl, such methyl, methoxymethyl, tetrahydropyranyl, benzyl, p-methoxybenzyl, trityl, and the like; alkoxycarbonyls, such as methoxycarbonyl, ethoxycarbonyl, isopropoxycarbonyl, n-isopropoxycarbonyl, n-butyloxycarbonyl, isobutyloxycarbonyl, sec-butyloxycarbonyl, t-butyloxycarbonyl, 2-ethylhexyloxycarbonyl, cyclohexyloxycarbonyl, methyloxycarbonyl, and the like; alkoxyalkoxycarbonyl groups, such as methoxymethoxycarbonyl, ethoxymethoxycarbonyl, 2-methoxyethoxycarbonyl, 2-ethoxyethoxycarbonyl, 2-butoxyethoxycarbonyl, 2-methoxyethoxymethoxycarbonyl, allyloxycarbonyl, propargyloxycarbonyl, 2-butenoxycarbonyl, 3-methyl-2-butenoxycarbonyl, and the like; haloalkoxycarbonyls, such as 2-chloroethoxycarbonyl, 2-chloroethoxycarbonyl, 2,2,2-trichloroethoxycarbonyl, and the like; optionally substituted arylalkoxycarbonyl groups, such as benzyloxycarbonyl, p-methylbenzyloxycarbonyl, p-methoxybenzyloxycarbonyl, p-nitrobenzyloxycarbonyl, 2,4-dinitrobenzyloxycarbonyl, 3,5-dimethylbenzyloxycarbonyl, p-chlorobenzyloxycarbonyl, p-bromobenzyloxy-carbonyl, fluorenylmethyloxycarbonyl, and the like; and optionally substituted aryloxycarbonyl groups, such as phenoxycarbonyl, p-nitrophenoxycarbonyl, o-nitrophenoxycarbonyl, 2,4-dinitrophenoxycarbonyl, p-methyl-phenoxycarbonyl, m-methylphenoxycarbonyl, o-bromophenoxycarbonyl, 3,5-dimethylphenoxycarbonyl, p-chlorophenoxycarbonyl, 2-chloro-4-nitrophenoxy-carbonyl, and the like); substituted alkyl, aryl, and alkaryl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl); carbonyl-protecting groups (e.g., acetal and ketal groups, such as dimethyl acetal, 1,3-dioxolane, and the like; acylal groups; and dithiane groups, such as 1,3-dithianes, 1,3-dithiolane, and the like); carboxylic acid-protecting groups (e.g., ester groups, such as methyl ester, benzyl ester, t-butyl ester, orthoesters, and the like; and oxazoline groups.

By “orthogonal overlap architecture” is meant a pair of double-stranded oligonucleotides where each overlap region of each double-stranded oligonucleotide is complementary to only the overlap region of the other double-stranded oligonucleotide. The complementary overlap regions may serve as a template for the ligation of the two oligonucleotides to increase ligation selectivity and efficiency. In particular, this architecture can allow for multiple tags to be added in the same reaction vessel (e.g., one-pot ligation) as the overlap regions template the ligation events between only tags with complementary overlap regions resulting in ligation selectivity.

The term “oxo” as used herein, represents ═O.

The prefix “perfluoro,” as used herein, represents alkyl group, as defined herein, where each hydrogen radical bound to the alkyl group has been replaced by a fluoride radical. For example, perfluoroalkyl groups are exemplified by trifluoromethyl, pentafluoroethyl, and the like. The term “protected hydroxyl,” as used herein, refers to an oxygen atom bound to an O-protecting group.

By “photo-reactive group” is meant a reactive group that participates in a reaction caused by absorption of ultraviolet, visible, or infrared radiation, thus producing a linkage. Exemplary, non-limiting photo-reactive groups are described herein.

By “primer” is meant an oligonucleotide that is capable of annealing to an oligonucleotide template and then being extended by a polymerase in a template-dependent manner.

By “protecting group” is a meant a group intended to protect the 3′-terminus or 5′-terminus of an oligonucleotide or to protect one or more functional groups of the chemical entity, scaffold, or building block against undesirable reactions during one or more binding steps of making, tagging, or using an oligonucleotide-encoded library. Commonly used protecting groups are disclosed in Greene, “Protective Groups in Organic Synthesis,” 4^{th} Edition (John Wiley & Sons, New York, 2007), which is incorporated herein by reference. Exemplary protecting groups for oligonucleotides include irreversible protecting groups, such as dideoxynucleotides and dideoxynucleosides (ddNTP or ddN), and, more preferably, reversible protecting groups for hydroxyl groups, such as ester groups (e.g., O-(α-methoxyethyl)ester, O-isovaleryl ester, and O-levulinyl ester), trityl groups (e.g., dimethoxytrityl and monomethoxytrityl), xanthenyl groups (e.g., 9-phenylxanthen-9-yl and 9-(p-methoxyphenyl)xanthen-9-yl), acyl groups (e.g., phenoxyacetyl and acetyl), and silyl groups (e.g., t-butyldimethylsilyl). Exemplary, non-limiting protecting groups for chemical entities, scaffolds, and building blocks include N-protecting groups to protect an amino group against undesirable reactions during synthetic procedure (e.g., acyl; aryloyl; carbamyl groups, such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries, such as protected or unprotected D, L or D, L-amino acids, such as alanine, leucine, phenylalanine, and the like; sulfonyl-containing groups, such as benzenesulfonyl, p-toluenesulfonyl, and the like; carbamate forming groups, such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5 dimethoxybenzyl oxycarbonyl, 2,4-dimethoxybenzyloxycarbonyl, 4 methoxybenzyloxycarbonyl, 2-nitro-4,5-dimethoxybenzyloxycarbonyl, 3,4,5 trimethoxybenzyloxycarbonyl, 1-(p-biphenylyl)-1-methylethoxycarbonyl, α,α-dimethyl-3,5 dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like; alkaryl groups, such as benzyl, triphenylmethyl, benzyloxymethyl, and the like; and silyl groups such as trimethylsilyl, and the like; where preferred N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz)); O-protecting groups to protect a hydroxyl group against undesirable reactions during synthetic procedure (e.g., alkylcarbonyl groups, such as acyl, acetyl, pivaloyl, and the like; optionally substituted arylcarbonyl groups, such as benzoyl; silyl groups, such as trimethylsilyl (TMS), tert-butyldimethylsilyl (TBDMS), tri-iso-propylsilyloxymethyl (TOM), triisopropylsilyl (TIPS), and the like; ether-forming groups with the hydroxyl, such methyl, methoxymethyl, tetrahydropyranyl, benzyl, p-methoxybenzyl, trityl, and the like; alkoxycarbonyls, such as methoxycarbonyl, ethoxycarbonyl, isopropoxycarbonyl, n-isopropoxycarbonyl, n-butyloxycarbonyl, isobutyloxycarbonyl, sec-butyloxycarbonyl, t-butyloxycarbonyl, 2-ethylhexyloxycarbonyl, cyclohexyloxycarbonyl, methyloxycarbonyl, and the like; alkoxyalkoxycarbonyl groups, such as methoxymethoxycarbonyl, ethoxymethoxycarbonyl, 2-methoxyethoxycarbonyl, 2-ethoxyethoxycarbonyl, 2-butoxyethoxycarbonyl, 2-methoxyethoxymethoxycarbonyl, allyloxycarbonyl, propargyloxycarbonyl, 2-butenoxycarbonyl, 3-methyl-2-butenoxycarbonyl, and the like; haloalkoxycarbonyls, such as 2-chloroethoxycarbonyl, 2-chloroethoxycarbonyl, 2,2,2-trichloroethoxycarbonyl, and the like; optionally substituted arylalkoxycarbonyl groups, such as benzyloxycarbonyl, p-methylbenzyloxycarbonyl, p-methoxybenzyloxycarbonyl, p-nitrobenzyloxycarbonyl, 2,4-dinitrobenzyloxycarbonyl, 3,5-dimethylbenzyloxycarbonyl, p-chlorobenzyloxycarbonyl, p-bromobenzyloxy-carbonyl, and the like; and optionally substituted aryloxycarbonyl groups, such as phenoxycarbonyl, p-nitrophenoxycarbonyl, o-nitrophenoxycarbonyl, 2,4-dinitrophenoxycarbonyl, p-methyl-phenoxycarbonyl, m-methylphenoxycarbonyl, o-bromophenoxycarbonyl, 3,5-dimethylphenoxycarbonyl, p-chlorophenoxycarbonyl, 2-chloro-4-nitrophenoxy-carbonyl, and the like); carbonyl-protecting groups (e.g., acetal and ketal groups, such as dimethyl acetal, 1,3-dioxolane, and the like; acylal groups; and dithiane groups, such as 1,3-dithianes, 1,3-dithiolane, and the like); carboxylic acid-protecting groups (e.g., ester groups, such as methyl ester, benzyl ester, t-butyl ester, orthoesters, and the like; silyl groups, such as trimethylsilyl, as well as any described herein; and oxazoline groups); and phosphate-protecting groups (e.g., optionally substituted ester groups, such as methyl ester, isopropyl ester, 2-cyanoethyl ester, allyl ester, t-butyl ester, benzyl ester, fluorenylmethyl ester, 2-(trimethylsilyl)ethyl ester, 2-(methylsulfonyl)ethyl ester, 2,2,2-trichloroethyl ester, 3′,5′-dimethoxybenzoin ester, p-hydroxyphenacyl ester, and the like).

By “proximity” or “in proximity” to a terminus of an oligonucleotide is meant near or closer to the stated terminus than the other remaining terminus. For example, a moiety or group in proximity to the 3′-terminus of an oligonucleotide is near or closer to the 3′-terminus than the 5′-terminus. In particular embodiments, a moiety or group in proximity to the 3′-terminus of an oligonucleotide is within one, two, three, four, five, six, seven, eight, nine, ten, fifteen, or more nucleotides from the 3′-terminus. In other embodiments, a moiety or group in proximity to the 5′-terminus of an oligonucleotide is within one, two, three, four, five, six, seven, eight, nine, ten, fifteen, or more nucleotides from the 5′-terminus.

By “purifying” is meant removing any unreacted product or any agent present in a reaction mixture that may reduce the activity of a chemical or biological agent to be used in a successive step. Purifying can include one or more of chromatographic separation, electrophoretic separation, and precipitation of the unreacted product or reagent to be removed.

By “relay primer” is meant an oligonucleotide that is capable of annealing to an oligonucleotide template that contains, in the region of the template to which the primer is hybridized, at least one internucleotide linkage that reduces the ability of a polymerase to read or translocate through. Upon hybridization, one or more relay primers allow for extension by a polymerase in a template dependent manner.

By “recombination,” as used herein, is meant the generation of a polymerase product as a result of at least two distinct hybridization events.

By “reversible immobilization” is meant immobilization of a conjugate or encoded chemical entityin a manner which allows for detachment from the support under gentle conditions (e.g., adsorption, ionic binding, affinity binding, chelation, disulfide bond formation, oligonucleotide hybridization, small molecule-small molecule interactions, reversible chemistry, protein-protein interactions, and hydrophobic interactions).

By “small molecule” drug or “small molecule” drug candidate is meant a molecule that has a molecular weight below about 1,000 Daltons. Small molecules may be organic or inorganic, isolated (e.g., from compound libraries or natural sources), or obtained by derivatization of known compounds.

The term “spirocyclyl,” as used herein, represents a C₂₋₇ alkylene diradical, both ends of which are bonded to the same carbon atom of the parent group to form a spirocyclic group, and also a C₁₋₆ heteroalkylene diradical, both ends of which are bonded to the same atom. The heteroalkylene radical forming the spirocyclyl group can containing one, two, three, or four heteroatoms independently selected from the group consisting of nitrogen, oxygen, and sulfur. In some embodiments, the spirocyclyl group includes one to seven carbons, excluding the carbon atom to which the diradical is attached. Spirocyclyl groups may be optionally substituted with 1, 2, 3, or 4 substituents provided herein as optional substituents for cycloalkyl and/or heterocyclyl groups.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers and/or conformers of the basic molecular structure. Some compounds of the present invention may exist in different tautomeric forms, all of the latter being included within the scope of the present invention.

By “substantially” is meant the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

By “substantial identity” or “substantially identical” is meant a polypeptide or polynucleotide sequence that has the same polypeptide or polynucleotide sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, or 20 contiguous amino acids, more preferably at least 25, 50, 75, 90, 100, 150, 200, 250, 300, or 350 contiguous amino acids, and most preferably the full-length amino acid sequence. For nucleic acids, the length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

By “sulfhydryl-reactive” is meant a group which exhibits reactivity with sulfhydryl groups, i.e., —SH. Exemplary, non-limiting sulfhydryl-reactive groups include haloacetyl, maleimide, aziridine, acryloyl, alkene (e.g., α,β-unsaturated carbonyl or vinylsulfone), and disulfide (e.g., pyridyl disulfide).

The term “sulfonyl,” as used herein, represents an —S(O)₂— group.

By “tag” or “oligonucleotide tag” is meant an oligonucleotide at least part of which encodes information. Non-limiting examples of such information include the addition (e.g., by a binding reaction) of a component (i.e., a scaffold or a building block, as in a scaffold tag or a building block tag, respectively), the headpiece in the library, the identity of the library (i.e., as in an identity tag), the use of the library (i.e., as in a use tag), and/or the origin of a library member (i.e., as in an origin tag).

By “tailpiece” is meant an oligonucleotide portion of the library that is attached to the conjugate or encoded chemical entity after the addition of all of the preceding tags and encodes for the identity of the library, the use of the library, and/or the origin of a library member.

The term “thiol,” as used herein. represents an —SH group.

By “triazole-forming” is meant a group (e.g., an optionally substituted alkynyl group) that reacts with a second triazole-forming group (e.g., an optionally substituted azido group) in a reaction (e.g., Huisgen 1,3-dipolar cycloaddition) to form a triazole group.

By “volatile” is meant easily evaporated at about 25° C. (e.g., about 20-30° C.) at atmospheric pressure or at a pressure less than atmospheric pressure. An example of a volatile compound is a compound having a boiling point between 15° C. and 100° C. (e.g., between 15° C. and 50° C., between 20° C. and 50° C., between 25° C. and 50° C., or between 30° C. and 50° C.). A mixture including a volatile compound can be separated by evaporating the volatile compound, leaving behind the less volatile compound or compounds.

Other features and advantages will be apparent from the following Detailed Description and the claims.

### BRIEF DESCRIPTION OF THE DRAWINGS

FIG. 1A and FIG. 1B show the LCMS of purified DBCO-HP006.

FIG. 2A and FIG. 2B show the LCMS of tamoxifen conjugated to Linker 1 and DBCO-HP006.

FIG. 3A and FIG. 3B show the LCMS of elacestrant (RAD1901) conjugated to Linker 1 and DBCO-HP006.

FIG. 4A and FIG. 4B show the LCMS of bazedoxifene conjugated to Linker 1 and DBCO-HP006.

FIG. 5A and FIG. 5B show the LCMS of 17β-estradiol conjugated to Linker 1 and DBCO-HP006.

FIG. 6A and FIG. 6B show the LCMS of (Z)-4-hydroxy tamoxifen conjugated to Linker 1 and DBCO-HP006.

FIG. 7A and FIG. 7B show the LCMS of 1,3,5-Tris(4-hydroxyphenyl)-4-propyl-1H-pyrazole (PPT) conjugated to Linker 1 and DBCO-HP006.

FIG. 8A and FIG. 8B show the LCMS of 1,3-bis(4-hydroxyphenyl)-4-methyl-5-[4-(2-piperidinylethoxy)phenol]-1H-pyrazole (MPP) conjugated to Linker 1 and DBCO-HP006.

FIG. 9A and FIG. 9B show the LCMS of WAY 200070 conjugated to Linker 1 and DBCO-HP006.

FIG. 10A and FIG. 10B show the LCMS of estriol conjugated to Linker 1 and DBCO-HP006.

FIG. 11A and FIG. 11B show the LCMS of diarylpropionitrile (DPN) conjugated to Linker 1 and DBCO-HP006.

FIG. 12 illustrates the product of one-pot ligation of an oligonucleotide headpiece, a headpiece extension, and four tags and shows the gel image of the product.

### DETAILED DESCRIPTION

The disclosure features a method of tagging large libraries of pre-existing compounds, e.g., libraries containing millions of individual compounds, with oligonucleotide tags in order to encode each member of the libraries with identifying information. The resulting encoded libraries can then be screened against targets (e.g., therapeutic targets such as proteins) as a mixture of the individual encoded compounds. This enables a robust and rapid method for identifying compounds of interest (e.g., drug leads, drug candidates, and/or tool compounds).

### Encoded Chemical Entities

This invention features encoded chemical entities including chemical entities (e.g., pre-existing chemical entities), bifunctional linkers, one or more oligonucleotide tags, and headpieces operatively associated with (i) the chemical entities via the bifunctional linkers; and (ii) the one or more oligonucleotide tags. Libraries of encoded chemical entities including chemical entities, bifunctional linkers, one or more oligonucleotide tags, and headpieces are further described below.

### Chemical Entities

The libraries of pre-existing chemical entities (e.g., compounds) or members can include one or more unique compounds.

### Bifunctional Linkers

The bifunctional linker between the headpiece and a chemical entity can be varied to provide an appropriate linking moiety and/or to increase the solubility of the headpiece in organic solvent. A wide variety of linkers are commercially available that can couple the headpiece with the small molecule library. The bifunctional linker typically consists of linear or branched chains and may include a C₁₋₁₀ alkyl, a heteroalkyl of 1 to 10 atoms, a C₂₋₁₀ alkenyl, a C₂₋₁₀ alkynyl, C₅₋₁₀ aryl, a cyclic or polycyclic system of 3 to 20 atoms, a phosphodiester, a peptide, an oligosaccharide, an oligonucleotide, an oligomer, a polymer, or a poly alkyl glycol (e.g., a poly ethylene glycol, such as —(CH₂CH₂O)ₙCH₂CH₂—, where n is an integer from 1 to 50), or combinations thereof.

The bifunctional linker may provide an appropriate linking moiety between the headpiece and a chemical entity of the library. In certain embodiments, the bifunctional linker includes three parts. Part 1 may be a reactive group, which forms a covalent bond with DNA, such as, e.g., a carboxylic acid, preferably activated by a N-hydroxy succinimide (NHS) ester to react with an amino group on the DNA (e.g., amino-modified dT), an amidite to modify the 5′ or 3′-terminus of a single-stranded headpiece (achieved by means of standard oligonucleotide chemistry), chemical-reactive pairs (e.g., azido-alkyne cycloaddition optionally in the presence of Cu(I) catalyst, or any described herein), or thiol reactive groups. Part 2 may also be a reactive group, which forms a covalent bond with the chemical entity, either building block Aₙ or a scaffold. Such a reactive group are, e.g., an amine, a thiol, an azide, or an alkyne. Part 3 may be a chemically inert linking moiety of variable length, introduced between Part 1 and 2. Such a linking moiety can be a chain of ethylene glycol units (e.g., PEGs of different lengths), an alkane, an alkene, a polyene chain, or a peptide chain. The linker can contain branches or inserts with hydrophobic moieties (such as, e.g., benzene rings) to improve solubility of the headpiece in organic solvents, as well as fluorescent moieties (e.g. fluorescein or Cy-3) used for library detection purposes. Hydrophobic residues in the headpiece design may be varied with the linker design to facilitate library synthesis in organic solvents. For example, the headpiece and linker combination is designed to have appropriate residues wherein the octanol:water coefficient (P_{oct}) is from, e.g., 1.0 to 2.5.

Linkers can be empirically selected for a given small molecule library design, such that the library can be synthesized in organic solvent, for example, in 15%, 25%, 30%, 50%, 75%, 90%, 95%, 98%, 99%, or 100% organic solvent. The linker can be varied using model reactions prior to library synthesis to select the appropriate chain length that solubilizes the headpiece in an organic solvent. Exemplary linkers include those having increased alkyl chain length, increased polyethylene glycol units, branched species with positive charges (to neutralize the negative phosphate charges on the headpiece), or increased amounts of hydrophobicity (for example, addition of benzene ring structures).

Linkers may also be branched, where branched linkers are well known in the art and examples can consist of symmetric or asymmetric doublers or a symmetric trebler. See, for example, Newcome et al., Dendritic Molecules: Concepts, Synthesis, Perspectives, VCH Publishers (1996); Boussif et al., Proc. Natl. Acad. Sci. USA 92:7297-7301 (1995); and Jansen et al., Science 266:1226 (1994).

Linkers optionally include one or more cross-linking groups. Examples of cross-linking groups include azide, carbene precursor group, and alkyne.

### Cross-Linking Groups

A cross-linking group refers to a group comprising a reactive functional group capable of chemically attaching to specific functional groups (e.g., primary amines, sulfhydryls) on proteins or other molecules. Examples of cross-linking groups include sulfhydryl-reactive cross-linking groups (e.g., groups comprising maleimides, haloacetyls, pyridyldisulfides, thiosulfonates, or vinylsulfones), amine-reactive cross-linking groups (e.g., groups comprising esters such as NHS esters, imidoesters, and pentafluorophenyl esters, or hydroxymethylphosphine), carboxyl-reactive cross-linking groups (e.g., groups comprising primary or secondary amines, alcohols, or thiols), carbonyl-reactive cross-linking groups (e.g., groups comprising hydrazides or alkoxyamines), triazole-forming cross-linking groups (e.g., groups comprising azides or alkynes) or carbene-generating groups such as aziridines.

Examples of chemically reactive functional groups which may react with cross-linking groups include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

Examples of moieties which are sulfhydryl-reactive include α-haloacetyl compounds of the type XCH₂CO— (where X=Br, Cl, or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but may additionally be useful in coupling to amino groups under certain conditions. Reagents such as 2-iminothiolane (Traut et al., Biochemistry 12:3266 (1973)), which introduce a thiol group through conversion of an amino group, may be considered as sulfhydryl reagents if linking occurs through the formation of disulfide bridges.

Examples of reactive moieties which are amino-reactive include, for example, alkylating and acylating agents. Representative alkylating agents include:

(i) α-haloacetyl compounds, which show specificity towards amino groups in the absence of reactive thiol groups and are of the type XCH₂CO— (where X=Br, Cl, or I), for example, as described by Wong Biochemistry 24:5337 (1979);

(ii) N-maleimide derivatives, which may react with amino groups either through a Michael type reaction or through acylation by addition to the ring carbonyl group, for example, as described by Smyth et al., J. Am. Chem. Soc. 82:4600 (1960) and Biochem. J. 91:589 (1964);

(iii) aryl halides such as reactive nitrohaloaromatic compounds;

(iv) alkyl halides, as described, for example, by McKenzie et al., J. Protein Chem. 7:581 (1988);

(v) aldehydes and ketones capable of Schiff's base formation with amino groups, the adducts formed usually being stabilized through reduction to give a stable amine;

(vi) epoxide derivatives such as epichlorohydrin and bisoxiranes, which may react with amino, sulfhydryl, or phenolic hydroxyl groups;

(vii) chlorine-containing derivatives of s-triazines, which are very reactive towards nucleophiles such as amino, sufhydryl, and hydroxyl groups;

(viii) aziridines based on s-triazine compounds detailed above, e.g., as described by Ross, J. Adv. Cancer Res. 2:1 (1954), which react with nucleophiles such as amino groups by ring opening;

(ix) squaric acid diethyl esters as described by Tietze, Chem. Ber. 124:1215 (1991); and

(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include:

(i) isocyanates and isothiocyanates, particularly aromatic derivatives, which form stable urea and thiourea derivatives respectively;

(ii) sulfonyl chlorides, which have been described by Herzig et al., Biopolymers 2:349 (1964);

(iii) acid halides;

(iv) active esters such as nitrophenylesters or N-hydroxysuccinimidyl esters;

(v) acid anhydrides such as mixed, symmetrical, or N-carboxyanhydrides;

(vi) other useful reagents for amide bond formation, for example, as described by M. Bodansky, Principles of Peptide Synthesis, Springer-Verlag, 1984;

(vii) acylazides, e.g., wherein the azide group is generated from a preformed hydrazide derivative using sodium nitrite, as described by Wetz et al., Anal. Biochem. 58:347 (1974);

(viii) imidoesters, which form stable amidines on reaction with amino groups, for example, as described by Hunter and Ludwig, J. Am. Chem. Soc. 84:3491 (1962); and

(ix) haloheteroaryl groups such as halopyridine or halopyrimidine.

Aldehydes and ketones may be reacted with amines to form Schiff's bases, which may advantageously be stabilized through reductive amination. Alkoxylamino moieties readily react with ketones and aldehydes to produce stable alkoxyamines, for example, as described by Webb et al., in Bioconjugate Chem. 1:96 (1990).

Examples of reactive moieties which are “carboxyl-reactive” include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through O-acylurea formation followed by amide bond formation, may also be employed.

Exemplary cross-linking groups include 2′-pyridyldisulfide, 4′-pyridyldisulfide iodoacetyl, maleimide, thioesters, alkyldisulfides, alkylamine disulfides, nitrobenzoic acid disulfide, anhydrides, NHS esters, aldehydes, alkyl chlorides, alkynes, and azides.

### Headpiece

In the library, the headpiece operatively links each chemical entity to its encoding oligonucleotide tag. Generally, the headpiece is a starting oligonucleotide having two functional groups that can be further derivatized, where the first functional group operatively links the chemical entity (or a component thereof) to the headpiece and the second functional group operatively links one or more tags to the headpiece. A bifunctional linker can optionally be used as a linking moiety between the headpiece and the chemical entity.

The functional groups of the headpiece can be used to form a covalent bond with a component of the chemical entity and another covalent bond with a tag. The component can be any part of the small molecule, such as a scaffold having diversity nodes or a building block. Alternatively, the headpiece can be derivatized to provide a linker (i.e., a linking moiety separating the headpiece from the small molecule to be formed in the library) terminating in a functional group (e.g., a hydroxyl, amine, carboxyl, sulfhydryl, alkynyl, azido, or phosphate group), which is used to form the covalent linkage with a component of the chemical entity. The linker can be attached to the 5′-terminus, at one of the internal positions, or to the 3′-terminus of the headpiece. When the linker is attached to one of the internal positions, the linker can be operatively linked to a derivatized base (e.g., the C5 position of uridine) or placed internally within the oligonucleotide using standard techniques known in the art. Exemplary linkers are described herein.

The headpiece can have any useful structure. The headpiece can be, e.g., 1 to 100 nucleotides in length, preferably 5 to 20 nucleotides in length, and most preferably 5 to 15 nucleotides in length. The headpiece can be single-stranded or double-stranded and can consist of natural or modified nucleotides, as described herein. For example, the chemical moiety can be operatively linked to the 3′-terminus or 5′-terminus of the headpiece. In particular embodiments, the headpiece includes a hairpin structure formed by complementary bases within the sequence. For example, the chemical moiety can be operatively linked to the internal position, the 3′-terminus, or the 5′-terminus of the headpiece.

Generally, the headpiece includes a non-self-complementary sequence on the 5′- or 3′-terminus that allows for binding an oligonucleotide tag by polymerization, enzymatic ligation, or chemical reaction. The headpiece can allow for ligation of oligonucleotide tags and optional purification and phosphorylation steps. After the addition of the last tag, an additional adapter sequence can be added to the 5′-terminus of the last tag. Exemplary adapter sequences include a primer-binding sequence or a sequence having a label (e.g., biotin). In cases where many building blocks and corresponding tags are used (e.g., 100), a mix-and-split strategy may be employed during the oligonucleotide synthesis step to create the necessary number of tags. Such mix-and-split strategies for DNA synthesis are known in the art. The resultant library members can be amplified by PCR following selection for binding entities versus a target(s) of interest.

The oligonucleotide headpiece of the encoded chemical entity can optionally include one or more primer-binding sequences. For example, the headpiece has a sequence in the loop region of the hairpin that serves as a primer-binding region for amplification, where the primer-binding region has a higher melting temperature for its complementary primer (e.g., which can include flanking identifier regions) than for a sequence in the headpiece. In other embodiments, the encoded chemical entity includes two primer-binding sequences (e.g., to enable PCR) on either side of one or more tags that encode one or more building blocks. Alternatively, the headpiece may contain one primer-binding sequence on the 5′- or 3′-terminus. In other embodiments, the headpiece is a hairpin, and the loop region forms a primer-binding site or the primer-binding site is introduced through hybridization of an oligonucleotide to the headpiece on the 3′ side of the loop. A primer oligonucleotide, containing a region homologous to the 3′-terminus of the headpiece and carrying a primer-binding region on its 5′-terminus (e.g., to enable a PCR reaction) may be hybridized to the headpiece and may contain a tag that encodes a building block or the addition of a building block. The primer oligonucleotide may contain additional information, such as a region of randomized nucleotides, e.g., 2 to 16 nucleotides in length, which is included for bioinformatics analysis.

The headpiece can optionally include a hairpin structure, where this structure can be achieved by any useful method. For example, the headpiece can include complementary bases that form intermolecular base pairing partners, such as by Watson-Crick DNA base pairing (e.g., adenine-thymine and guanine-cytosine) and/or by wobble base pairing (e.g., guanine-uracil, inosine-uracil, inosine-adenine, and inosine-cytosine). In another example, the headpiece can include modified or substituted nucleotides that can form higher affinity duplex formations compared to unmodified nucleotides, such modified or substituted nucleotides being known in the art.

The oligonucleotide headpiece of the encoded chemical entity can optionally include one or more labels that allow for detection. For example, the headpiece, one or more oligonucleotide tags, and/or one or more primer sequences can include an isotope, a radioimaging agent, a marker, a tracer, a fluorescent label (e.g., rhodamine or fluorescein), a chemiluminescent label, a quantum dot, and a reporter molecule (e.g., biotin or a his-tag).

In other embodiments, the headpiece or tag may be modified to support solubility in semi-, reduced-, or non-aqueous (e.g., organic) conditions. Nucleotide bases of the headpiece or tag can be rendered more hydrophobic by modifying, for example, the C5 positions of T or C bases with aliphatic chains without significantly disrupting their ability to hydrogen bond to their complementary bases. Exemplary modified or substituted nucleotides are 5′-dimethoxytrityl-N4-diisobutylaminomethylidene-5-(1-propynyl)-2′-deoxycytidine,3′-[(2-cyanoethyl)-(N,N-diisopropyl)]-phosphoramidite; 5′-dimethoxytrityl-5-(1-propynyl)-2′-deoxyuridine,3′-[(2-cyanoethyl)-(N,N-diisopropyl)]-phosphoramidite; 5′-dimethoxytrityl-5-fluoro-2′-deoxyuridine,3′-[(2-cyanoethyl)-(N,N-diisopropyl)]-phosphoramidite; and 5′-dimethoxytrityl-5-(pyren-1-yl-ethynyl)-2′-deoxyuridine, or 3′-[(2-cyanoethyl)-(N,N-diisopropyl)]-phosphoramidite.

In addition, the headpiece oligonucleotide can be interspersed with modifications that promote solubility in organic solvents. For example, azobenzene phosphoramidite can introduce a hydrophobic moiety into the headpiece design. Such insertions of hydrophobic amidites into the headpiece can occur anywhere in the molecule. However, the insertion cannot interfere with subsequent tagging using additional DNA tags during the library synthesis or ensuing PCR once a selection is complete or microarray analysis, if used for tag deconvolution. Such additions to the headpiece design described herein would render the headpiece soluble in, for example, 15%, 25%, 30%, 50%, 75%, 90%, 95%, 98%, 99%, or 100% organic solvent. Thus, addition of hydrophobic residues into the headpiece design allows for improved solubility in semi- or non-aqueous (e.g., organic) conditions, while rendering the headpiece competent for oligonucleotide tagging. Furthermore, DNA tags that are subsequently introduced into the library can also be modified at the C5 position of T or C bases such that they also render the library more hydrophobic and soluble in organic solvents for subsequent steps of library synthesis.

In particular embodiments, the headpiece and the first tag can be the same entity, i.e., a plurality of headpiece-tag entities can be constructed that all share common parts (e.g., a primer-binding region) and all differ in another part (e.g., encoding region). These may be utilized in the “split” step and pooled after the event they are encoding has occurred.

In particular embodiments, the headpiece can encode information, e.g., by including a sequence that encodes the first split(s) step or a sequence that encodes the identity of the library, such as by using a particular sequence related to a specific library.

### Oligonucleotide Tags

The oligonucleotide tags described herein (e.g., a tag or a portion of a headpiece or a portion of a tailpiece) can be used to encode any useful information, such as a molecule, a portion of a chemical entity, the addition of a component (e.g., a scaffold or a building block), a headpiece in the library, the identity of the library, the use of one or more library members (e.g., use of the members in an aliquot of a library), and/or the origin of a library member (e.g., by use of an origin sequence).

Any sequence in an oligonucleotide can be used to encode any information. Thus, one oligonucleotide sequence can serve more than one purpose, such as to encode two or more types of information or to provide a starting oligonucleotide that also encodes for one or more types of information.

For example, the first tag can encode for the addition of a first building block, as well as for the identification of the library. In another example, a headpiece can be used to provide a starting oligonucleotide that operatively links a chemical entity to a tag, where the headpiece additionally includes a sequence that encodes for the identity of the library (i.e., the library-identifying sequence). Accordingly, any of the information described herein can be encoded in separate oligonucleotide tags or can be combined and encoded in the same oligonucleotide sequence (e.g., an oligonucleotide tag, such as a tag, or a headpiece).

A building block sequence encodes for the identity of a building block and/or the type of binding reaction conducted with a building block. This building block sequence is included in a tag, where the tag can optionally include one or more types of sequence described below (e.g., a library-identifying sequence, a use sequence, and/or an origin sequence).

A library-identifying sequence encodes for the identity of a particular library. In order to permit mixing of two or more libraries, a library member may contain one or more library-identifying sequences, such as in a library-identifying tag (i.e., an oligonucleotide including a library-identifying sequence), in a ligated tag, in a part of the headpiece sequence, or in a tailpiece sequence. These library-identifying sequences can be used to deduce encoding relationships, where the sequence of the tag is translated and correlated with chemical (synthesis) history information. Accordingly, these library-identifying sequences permit the mixing of two or more libraries together for selection, amplification, purification, sequencing, etc.

A use sequence encodes the history (i.e., use) of one or more library members in an individual aliquot of a library. For example, separate aliquots may be treated with different reaction conditions, building blocks, and/or selection steps. In particular, this sequence may be used to identify such aliquots and deduce their history (use) and thereby permit the mixing together of aliquots of the same library with different histories (uses) (e.g., distinct selection experiments) for the purposes of the mixing together of samples together for selection, amplification, purification, sequencing, etc. These use sequences can be included in a headpiece, a tailpiece, a tag, a use tag (i.e., an oligonucleotide including a use sequence), or any other tag described herein (e.g., a library-identifying tag or an origin tag).

An origin sequence is a degenerate (random, stochastically-generated) oligonucleotide sequence of any useful length (e.g., about six oligonucleotides) that encodes for the origin of the library member. This sequence serves to stochastically subdivide library members that are otherwise identical in all respects into entities distinguishable by sequence information, such that observations of amplification products derived from unique progenitor templates (e.g., selected library members) can be distinguished from observations of multiple amplification products derived from the same progenitor template (e.g., a selected library member). For example, after library formation and prior to the selection step, each library member can include a different origin sequence, such as in an origin tag. After selection, selected library members can be amplified to produce amplification products, and the portion of the library member expected to include the origin sequence (e.g., in the origin tag) can be observed and compared with the origin sequence in each of the other library members. As the origin sequences are degenerate, each amplification product of each library member should have a different origin sequence. However, an observation of the same origin sequence in the amplification product could indicate multiple amplicons derived from the same template molecule. When it is desired to determine the statistics and demographics of the population of encoding tags prior to amplification, as opposed to post-amplification, the origin tag may be used. These origin sequences can be included in a headpiece, a tailpiece, a tag, an origin tag (i.e., an oligonucleotide including an origin sequence), or any other tag described herein (e.g., a library-identifying tag or a use tag).

Any of the types of sequences described herein can be included in the headpiece. For example, the headpiece can include one or more of a building block sequence, a library-identifying sequence, a use sequence, or an origin sequence.

Any of these sequences described herein can be included in a tailpiece. For example, the tailpiece can include one or more of a library-identifying sequence, a use sequence, or an origin sequence.

Any of tags described herein can include a connector at or in proximity to the 5′- or 3′-terminus having a fixed sequence. Connectors facilitate the formation of linkages (e.g., chemical linkages) by providing a reactive group (e.g., a chemical-reactive group or a photo-reactive group) or by providing a site for an agent that allows for a linkage (e.g., an agent of an intercalating moiety or a reversible reactive group in the connector(s) or cross-linking oligonucleotide). Each 5′-connector may be the same or different, and each 3′-connector may be the same or different. In an exemplary, non-limiting conjugate or encoded chemical entity having more than one tags, each tag can include a 5′-connector and a 3′-connector, where each 5′-connector has the same sequence and each 3′-connector has the same sequence (e.g., where the sequence of the 5′-connector can be the same or different from the sequence of the 3′-connector). The connector provides a sequence that can be used for one or more linkages. To allow for binding of a relay primer or for hybridizing a cross-linking oligonucleotide, the connector can include one or more functional groups allowing for a linkage (e.g., a linkage for which a polymerase has reduced ability to read or translocate through, such as a chemical linkage).

These sequences can include any modification described herein for oligonucleotides, such as one or more modifications that promote solubility in organic solvents (e.g., any described herein, such as for the headpiece), that provide an analog of the natural phosphodiester linkage (e.g., a phosphorothioate analog), or that provide one or more non-natural oligonucleotides (e.g., 2′-substituted nucleotides, such as 2′-O-methylated nucleotides and 2′-fluoro nucleotides, or any described herein).

These sequences can include any characteristics described herein for oligonucleotides. For example, these sequences can be included in tag that is less than 20 nucleotides (e.g., as described herein). In other examples, the tags including one or more of these sequences have about the same mass (e.g., each tag has a mass that is about +/−10% from the average mass between within a specific set of tags that encode a specific variable); lack a primer-binding (e.g., constant) region; lack a constant region; or have a constant region of reduced length (e.g., a length less than 30 nucleotides, less than 25 nucleotides, less than 20 nucleotides, less than 19 nucleotides, less than 18 nucleotides, less than 17 nucleotides, less than 16 nucleotides, less than 15 nucleotides, less than 14 nucleotides, less than 13 nucleotides, less than 12 nucleotides, less than 11 nucleotides, less than 10 nucleotides, less than 9 nucleotides, less than 8 nucleotides, or less than 7 nucleotides).

Sequencing strategies for libraries and oligonucleotides of this length may optionally include concatenation or catenation strategies to increase read fidelity or sequencing depth, respectively. In particular, the selection of encoded libraries that lack primer-binding regions has been described in the literature for SELEX, such as described in Jarosch et al., Nucleic Acids Res. 34: e86 (2006), which is incorporated herein by reference. For example, a library member can be modified (e.g., after a selection step) to include a first adapter sequence on the 5′-terminus of the conjugate or encoded chemical entity and a second adapter sequence on the 3′-terminus of the conjugate or encoded chemical entity, where the first sequence is substantially complementary to the second sequence and result in forming a duplex. To further improve yield, two fixed dangling nucleotides (e.g., CC) are added to the 5′-terminus. In particular embodiments, the first adapter sequence is 5′-GTGCTGC-3′ (SEQ ID NO: 1), and the second adapter sequence is 5′-GCAGCACCC-3′ (SEQ ID NO: 2).

### Enzymatic Ligation and Chemical Ligation Techniques

Various ligation techniques can be used to add tags to the headpiece to produce an encoded chemical entity. Accordingly, any of the binding steps described herein can include any useful ligation techniques, such as enzymatic ligation and/or chemical ligation. These binding steps can include the addition of one or more tags to the oligonucleotide headpiece of the encoded chemical entity. In particular embodiments, the ligation techniques used for any oligonucleotide provide a resultant product that can be transcribed and/or reverse transcribed to allow for decoding of the library or for template-dependent polymerization with one or more DNA or RNA polymerases.

Generally, enzymatic ligation produces an oligonucleotide having a native phosphodiester bond that can be transcribed and/or reverse transcribed. Exemplary methods of enzyme ligation are provided herein and include the use of one or more RNA or DNA ligases, such as T4 RNA ligase 1 or 2, T4 DNA ligase, CircLigase™ ssDNA ligase, CircLigase™ II ssDNA ligase, and ThermoPhage™ ssDNA ligase (Prokazyme Ltd., Reykjavik, Iceland).

Chemical ligation can also be used to produce oligonucleotides capable of being transcribed or reverse transcribed or otherwise used as a template for a template-dependent polymerase. The efficacy of a chemical ligation technique to provide oligonucleotides capable of being transcribed or reverse transcribed may need to be tested. This efficacy can be tested by any useful method, such as liquid chromatography-mass spectrometry, RT-PCR analysis, PCR analysis, electrophoresis, and/or sequencing.

### Reaction Conditions to Promote Enzymatic Ligation or Chemical Ligation

The methods described herein can include one or more reaction conditions that promote enzymatic or chemical ligation between the headpiece and a tag or between two tags. These reaction conditions include using modified nucleotides within the tag, as described herein; using donor tags and acceptor tags having different lengths and varying the concentration of the tags; using different types of ligases, as well as combinations thereof (e.g., CircLigase™ DNA ligase and/or T4 RNA ligase), and varying their concentration; using poly ethylene glycols (PEGs) having different molecular weights and varying their concentration; use of non-PEG crowding agents (e.g., betaine or bovine serum albumin); varying the temperature and duration for ligation; varying the concentration of various agents, including ATP, Co(NH₃)₆Cl₃, and yeast inorganic pyrophosphate; using enzymatically or chemically phosphorylated oligonucleotide tags; using 3′-protected tags; and using preadenylated tags. These reaction conditions also include chemical ligations.

The headpiece and/or tags can include one or more modified or substituted nucleotides. In preferred embodiments, the headpiece and/or tags include one or more modified or substituted nucleotides that promote enzymatic ligation, such as 2′-O-methyl nucleotides (e.g., 2′-O-methyl guanine or 2′-O-methyl uracil), 2′-fluoro nucleotides, or any other modified nucleotides that are utilized as a substrate for ligation. Alternatively, the headpiece and/or tags are modified to include one or more chemically reactive groups to support chemical ligation (e.g. an optionally substituted alkynyl group and an optionally substituted azido group). Optionally, the tag oligonucleotides are functionalized at both termini with chemically reactive groups, and, optionally, one of these termini is protected, such that the groups may be addressed independently and side-reactions may be reduced (e.g., reduced polymerization side-reactions).

As described herein, chemical ligation which results in phosphodiester, phosphonate, or phosphorothioate linkages may be performed by reaction of a 5′- or 3′-phosphate, phosphonate, or phosphorothioate with a 5′- or 3′-hydroxyl group in the presence of cyanoimidazole and a divalent metal ion such as Zn²⁺.

Enzymatic ligation can include one or more ligases. Exemplary ligases include CircLigase™ ssDNA ligase (EPICENTRE Biotechnologies, Madison, Wis.), CircLigase™ II ssDNA ligase (also from EPICENTRE Biotechnologies), ThermoPhage™ ssDNA ligase (Prokazyme Ltd., Reykjavik, Iceland), T4 RNA ligase, and T4 DNA ligase. In preferred embodiments, ligation includes the use of an RNA ligase or a combination of an RNA ligase and a DNA ligase. Ligation can further include one or more soluble multivalent cations, such as Co(NH₃)₆Cl₃, in combination with one or more ligases.

Before or after the ligation step, a conjugate or encoded chemical entity can be purified. In some embodiments, the conjugate or encoded chemical entity can be purified to remove unreacted headpiece or tags that may result in cross-reactions and introduce “noise” into the encoding process. In some embodiments, the conjugate or encoded chemical entity can be purified to remove any reagents or unreacted starting material that can inhibit or lower the ligation activity of a ligase. For example, orthophosphate may result in lowered ligation activity. In certain embodiments, entities that are introduced into a chemical or ligation step may need to be removed to enable the subsequent chemical or ligation step. Methods of purifying the conjugate or encoded chemical entity are described herein. Purification of the conjugate or encoded chemical entity may be carried out by reversible immobilization of the conjugate or encoded chemical entity followed by purification and release prior to a subsequent step.

Enzymatic and chemical ligation can include poly ethylene glycol having an average molecular weight of more than 300 Daltons (e.g., more than 600 Daltons, 3,000 Daltons, 4,000 Daltons, 5,000, 6,000, 7,000, 8,000, 9,000, 10,000, 15,000, 20,000, 25,000, 30,000, 35,000, 40,000, or 45,000 Daltons). In particular embodiments, the polyethylene glycol has an average molecular weight from about 3,000 Daltons to 9,000 Daltons (e.g., from 3,000 Daltons to 8,000 Daltons, from 3,000 Daltons to 7,000 Daltons, from 3,000 Daltons to 6,000 Daltons, and from 3,000 Daltons to 5,000 Daltons). In preferred embodiments, the poly ethylene glycol has an average molecular weight from about 3,000 Daltons to about 6,000 Daltons (e.g., from 3,300 Daltons to 4,500 Daltons, from 3,300 Daltons to 5,000 Daltons, from 3,300 Daltons to 5,500 Daltons, from 3,300 Daltons to 6,000 Daltons, from 3,500 Daltons to 4,500 Daltons, from 3,500 Daltons to 5,000 Daltons, from 3,500 Daltons to 5,500 Daltons, and from 3,500 Daltons to 6,000 Daltons, such as 4,600 Daltons). Polyethylene glycol can be present in any useful amount, such as from about 25% (w/v) to about 35% (w/v), such as 30% (w/v).

### Methods for Tagging Encoded Libraries

The methods described herein can be used to synthesize libraries having a diverse number of chemical entities that are encoded by oligonucleotide tags. The invention features methods for operatively associating oligonucleotide tags with chemical entities (e.g., compounds such as pre-existing compounds), such that encoding relationships may be established between the sequence of the tag and the identity of the chemical entity. In particular, the identity of a chemical entity can be inferred from the sequence of bases in the oligonucleotide. Using this method, a library including diverse chemical entities can be encoded with a particular set of tags.

Generally, these methods include the use of i) a chemical entity; ii) a bifunctional linker including a carbene precursor group and a cross-linking group; iii) a conjugate including an oligonucleotide headpiece and a cross-linking group; and iv) an oligonucleotide tag or unique combination of tags designed to ligate to each other. One oligonucleotide tag is bound to the oligonucleotide headpiece. Binding can be effectuated by any useful means, such as by enzymatic binding (e.g., ligation with one or more of an RNA ligase and/or a DNA ligase) or by chemical binding (e.g., by a substitution reaction between two functional groups, such as a nucleophile and a leaving group).

This invention describes a practical method of encoding millions of individual chemical entities (e.g., pre-existing compounds) using unique combinations of encoding oligonucleotides. As an example, an encoding strategy in which each final concatenated tag set has the design Compound-Linker-Headpiece-TagA-TagB-TagC-TagD-Tailpiece can uniquely encode 6.25 million (50×50×50×50) compounds with one oligonucleotide Headpiece, 50 unique oligonucleotide TagA's, 50 unique oligonucleotide TagB's, 50 unique oligonucleotide TagC's, 50 oligonucleotide TagD's, and one oligonucleotide Tailpiece. This totals 200 unique oligonucleotide tags, one oligonucleotide Headpiece, and one oligonucleotide Tailpiece. The Headpiece and Tailpiece can contain constant primer-binding sequences or provide a functional group to allow for binding (e.g., by ligation) of a primer-binding sequence that are used for amplification and optionally are utilized for clustering and sequencing. The primer-binding sequence can be used for amplifying and/or sequencing the oligonucleotides tags of the conjugate or encoded chemical entity. Exemplary methods for amplifying and for sequencing include polymerase chain reaction (PCR), linear chain amplification (LCR), rolling circle amplification (RCA), or any other method known in the art to amplify or determine nucleic acid sequences. Dispensing well-specific combinations of these oligonucleotide tags along with the individual compounds that they will encode is readily automated.

The oligonucleotide tags may be single-stranded or double-stranded and contain orthogonal ligation overlaps that allow them to ligate in a precise spatial order even if all oligonucleotides are introduced simultaneously into a “one-pot” reaction mixture. Oligonucleotides are appropriately modified for ligation (e.g., by 5′-phosphorylation).

### Methods for Screening Encoded Libraries

Next, the library can be tested and/or selected for a characteristic or function, as described herein. For example, the mixture of tagged chemical entities can be separated into at least two populations, where the first population is enriched for members that bind to a particular biological target and the second population that is less enriched (e.g., by negative selection or positive selection). The first population can then be selectively captured (e.g., by eluting from a column providing the target of interest or by incubating the aliquot with the target of interest followed by capture of the protein along with associated library members and subsequent elution of library members) and, optionally, further analyzed or tested, such as with optional washing, purification, negative selection, positive selection, or separation steps. Adaptation of these methods can yield reversible or irreversible covalent target modifiers when a library elution step is included that cleaves at least one covalent bond, either within or between the encoding tags of the library member and the matrix or within the target protein, for example using a restriction endonuclease or a protease.

Once the pre-existing compounds from the first library that bind to the target of interest have been identified, a second library of pre-existing compounds may be encoded and screened against targets of interest.

### Methods for Decoding Encoded Libraries

Finally, the identity of the encoded chemical entities within a selected population can be determined by the sequence of the oligonucleotide tags. Upon correlating the sequence with encoded library members tagging history, this method can identify the individual members of the library with the selected characteristic (e.g., an increased tendency to bind to the target protein and thereby elicit a therapeutic effect). For further testing and optimization, candidate therapeutic compounds may then be prepared by synthesizing the identified library members with or without their associated oligonucleotide tags or by directly accessing individual pre-existing compounds that were used to construct the library, either with or without modification by a reactive or photoreactive linker element.

The methods described herein can include any number of optional steps to diversify the library or to interrogate the members of the library. For any tagging method described herein, successive “n” number of tags can be added with additional “n” number of ligation, separation, and/or phosphorylation steps or alternatively with “successive” ligations occurring in a “single-pot” reaction to provide a unique combinatorial catenated tag set. Exemplary optional steps include restriction of library member-associated encoding oligonucleotides using one or more restriction endonucleases; repair of the associated encoding oligonucleotides, e.g., with any repair enzyme, such as those described herein; ligation of one or more adapter sequences to one or both of the termini for library member-associated encoding oligonucleotides, e.g., such as one or more adapter sequences to provide a priming sequence for amplification and sequencing or to provide a label, such as biotin, for immobilization of the sequence; reverse-transcription or transcription, optionally followed by reverse-transcription, of the assembled tags in the conjugate or encoded chemical entity using a reverse transcriptase, transcriptase, or another template-dependent polymerase; amplification of the assembled tags in the conjugate or encoded chemical entity using, e.g., PCR; generation of clonal isolates of one or more populations of assembled tags in the conjugate or encoded chemical entity, e.g., by use of bacterial transformation, emulsion formation, dilution, surface capture techniques, etc.; amplification of clonal isolates of one or more populations of assembled tag in the conjugate or encoded chemical entity, e.g., by using clonal isolates as templates for template-dependent polymerization of nucleotides; and sequence determination of clonal isolates of one or more populations of assembled tags in the conjugate or encoded chemical entity, e.g., by using clonal isolates as templates for template-dependent polymerization with fluorescently labeled nucleotides with reversible terminator chemistry. Additional methods for amplifying and sequencing the oligonucleotide tags are described herein.

These methods can be used to identify and discover any number of chemical entities with a particular characteristic or function, e.g., in a selection step. The desired characteristic or function may be used as the basis for partitioning the library into at least two parts with the concomitant enrichment of at least one of the members or related members in the library with the desired function. In particular embodiments, the method comprises identifying a small drug-like library member that binds or inactivates a protein of therapeutic interest. In any of these instances, the oligonucleotide tags encode the chemical history of the library member and in each case a collection of chemical possibilities may be represented by any particular tag combination.

In one embodiment, the library of chemical entities, or a portion thereof, is contacted with a biological target under conditions suitable for at least one member of the library to bind to the target, followed by removal of library members that do not bind to the target, and analyzing the one or more oligonucleotide tags associated with the target. This method can optionally include amplifying the tags by methods known in the art. Exemplary biological targets include enzymes (e.g., kinases, phosphatases, methylases, demethylases, proteases, and DNA repair enzymes), proteins involved in protein:protein interactions (e.g., ligands for receptors), receptor targets (e.g., GPCRs and RTKs), ion channels, bacteria, viruses, parasites, DNA, RNA, prions, and carbohydrates.

In another embodiment, the encoded chemical entities that bind to a target are not subjected to amplification but are analyzed directly. Exemplary methods of analysis include microarray analysis, including evanescent resonance photonic crystal analysis; bead-based methods for deconvoluting tags (e.g., by using his-tags); label-free photonic crystal biosensor analysis (e.g., a BIND® Reader from SRU Biosystems, Inc., Woburn, Mass.); or hybridization-based approaches (e.g. by using arrays of immobilized oligonucleotides complementary to sequences present in the library of tags).

In addition, chemical-reactive pairs can be readily included in solid-phase oligonucleotide synthesis schemes and will support the efficient chemical ligation of oligonucleotides. In addition, the resultant ligated oligonucleotides can act as templates for template-dependent polymerization with one or more polymerases. Accordingly, any of the binding steps described herein for tagging encoded libraries can be modified to include one or more of enzymatic ligation and/or chemical ligation techniques. Exemplary ligation techniques include enzyme ligation, such as use of one of more RNA ligases and/or DNA ligases; and chemical ligation, such as use of chemical-reactive pairs (e.g., a pair including optionally substituted alkynyl and azido functional groups).

In some embodiments, amplifying can optionally include forming a water-in-oil emulsion to create a plurality of aqueous microreactors. The reaction conditions (e.g., concentration of conjugate or encoded chemical entity and size of microreactors) can be adjusted to provide, on average, a microreactor having at least one member of a library of compounds. Each microreactor can also contain the target, a single bead capable of binding to an encoded chemical entity or a portion of the encoded chemical entity (e.g., one or more tags) and/or binding the target, and an amplification reaction solution having one or more necessary reagents to perform nucleic acid amplification. After amplifying the tag in the microreactors, the amplified copies of the tag will bind to the beads in the microreactors, and the coated beads can be identified by any useful method.

### General Strategy for Tagging, Screening, and Decoding Encoded Libraries of Pre-Existing Chemical Entities

The methods described herein may involve introduction of an entire library of chemical entities (e.g., compound collection) as individual chemical entities (e.g., compounds) into each well on a one-compound, one-well basis, similar to commonly utilized processes for the generation of assay-ready plates. This may be followed by the introduction of a bifunctional linker (e.g., 3-(2-Azidoethyl)-3-methyl-3H-diazirine) at high relative concentration in an organic solvent followed by irradiation to activate the aziridine group and allow for the formation of a covalent linkage between the bifunctional linker and the compound to be encoded. Subsequent reduction of pressure may remove excess unreacted bifunctional linker and optionally all or some of the organic solvent. In the succeeding step a bifunctional headpiece oligonucleotide may be introduced into each well along with a well-specific combination of encoding tags, a ligase enzyme and a ligase-competent buffer. The encoding tags are designed to ligate to the headpiece and to each other in a precisely determined order by careful design of their ligation junctions. The headpiece also contains a strained alkyne that will react with the azide that is connected to the compound to be encoded in a copper-free click reaction since copper may interfere with the ligation efficiency or specificity.

Subsequently the contents of the individual wells may be quenched, combined and then further purified and concentrated as a mixture before ligation to a tailpiece containing a library-identifying encoding sequence along with other tag sequences as desired. Once generated aliquots of the library may be used for affinity-mediated screening either combined with other encoded libraries or not.

The one-pot ligation of a well-specific combination of tags allows for the tagging of larger libraries of pre-existing compounds (e.g., libraries of millions of compounds rather than libraries of thousands of compounds). Additionally, the present invention allows for incubating the pre-existing compounds with volatile diazirine-azide linker that upon irradiation can insert the resulting carbene into potentially multiple reactive sites on the compounds. Furthermore, this method allows for the unreacted cross-linker to be removed at low pressure, followed by conjugation of the azide to the headpiece. HTS-ready plates of libraries of pre-existing compounds are encoded with well-specific combinations of oligonucleotide tags via a single ligation.

Traditional HTS utilizes activity-based discovery of target-modulating molecules by detecting their influence upon assays with readouts derived from biochemical (e.g. enzymatic transformation of substrates), biophysical (e.g. labeled probe displacement) or biological (e.g. cell-based). Generally these assays are conducted with a low concentration of target (e.g. protein) and a high concentration of a putative target-modulating molecule (e.g., a small molecule compound that is part of a library of pre-existing compounds). Such screens are to a great extent confounded by artifacts that result from the high concentration of the small molecule such as aggregation-mediated or insolubility-mediated signal.

The opportunity to run an affinity-mediated screen on the same library of compounds but encoded by oligonucleotide tags provides an opportunity to determine which compound collection members interact with the target protein under an entirely distinct assay environment (e.g., the individual compound concentrations are low). Furthermore, solubility is conferred by the conjugated oligonucleotides, thereby offering orthogonal assay data that aids in the identification of genuine hits from the original screen. In many cases, more than half of the timeline of a project utilizing a pre-existing combinatorially-generated DNA-encoded chemical library is dedicated to the re-synthesis of off-DNA versions of the molecules enriched in the affinity-mediated library screen. Thus, the encoding of libraries of pre-existing compounds accelerates project timelines since no re-synthesis of enriched compounds identified in the screen is necessary since all compounds pre-exist within the original library or collection.

The library of pre-existing compounds may be a collection of compounds utilized by pharmaceutical companies to discover modulators of target proteins. The individual members of the collection may be aliquoted into separate compartments (e.g., individual wells of multiwall plates (e.g. 96-well plates, 384-well plates, or 1536-well plates)). Each compound within each well may be reacted by incubation with a linker, for example a volatile bifunctional linker. An example of a volatile bifunctional linker is a low molecular weight compound which includes a diazirene group (a carbene precursor) and an azide group (a cross-linking group). The diazirene functional group is reacted with the compound under suitable reaction conditions (e.g., photochemical conditions via irradiation). Irradiation activates the diazirine group, transforming it into a carbene. Photochemically activated diazirines can insert themselves into a range of covalent bonds, thereby forming covalent linkages to molecules not designed with conjugation in mind, and because they can react at multiple loci within individual molecules they can display them from multiple vectors allowing for the discovery of molecules that are inactivated by conjugation at some positions. Reduced pressure can then be used to remove the volatile unreacted bifunctional linker and the residual functionalized HTS compound can then be conjugated to an azide-reactive oligonucleotide and then encoded by the introduction of a combination of oligonucleotides that have been designed to ligate to each other and to the azide-reactive oligonucleotide in a defined order to generate an amplifiable concatenated set of oligonucleotide tags and primer-binding sequences. An example of a suitable volatile bifunctional cross-linker is 3-(2-Azidoethyl)-3-methyl-3H-diazirine.

The individual amplifiable encoded oligonucleotide-HTS deck compounds can be combined, optionally further purified and concentrated as a mixture, and subjected to affinity-mediated screens followed by polymerase-mediated amplification and sequencing to identify enriched library members. Confirmation of the target-modulating activity of individual enriched HTS deck compounds may then be established by testing individual HTS deck compounds in their off-DNA form in appropriate activity assays. There is no need to resynthesize the untagged compounds since they already exist.

### EXAMPLES

### Example 1—Tagging Pre-Existing Compounds

Reacting a Chemical Entity with a Bifunctional Linker Including a Carbene Precursor Group and a First Cross-Linking Group to Produce a First Conjugate

Chemical entities are sourced from libraries of pre-existing compounds and aliquoted into multiwell plates with one compound per well. These may be in solution or dry and may be placed in 96-well, 384-well or 1536-well or other spatially segregated compartments.

A bifunctional linker (e.g., a volatile bifunctional linker (VBL)) is synthesized or obtained commercially. One reactive group of the VBL can be photochemically reacted to produce a carbene. The other reactive group is an azide cross-linking group suitable for click chemistry. An example of a VBL is:

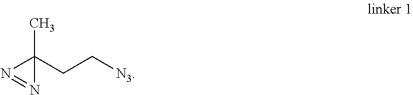

The synthesis of linker 1 (3-(2-Azidoethyl)-3-methyl-3H-diazirine) is reported in Liang et al., Angew. Chem. Int. Ed. Engl. 56 (10):2744-2748 (2017).

Then linker 1 and dimethylsulfoxide (DMSO) are added to each well of the succession of multiwell plates, or other spatially addressable compartments, and irradiated at 365 nm for 30 minutes. The resulting first conjugate in each well is purified by removing unreacted linker 1 by evaporation under reduced pressure (e.g., about 400 torr) and elevated temperature (e.g., about 25-30° C.).

Each first conjugate has the structure of the following:

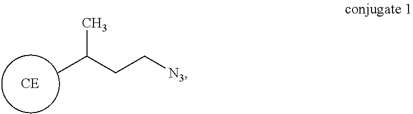

where CE represents a structure including a chemical entity.

Synthesis of a Second Conjugate Including an Oligonucleotide Headpiece and a Cross-Linking Group

A second conjugate including an oligonucleotide headpiece and a cross-linking group is synthesized from a primary amine-terminated oligonucleotide headpiece and a linker including a dibenzocyclooctyne-amino (DBCO) group. An example of an amine-terminated oligonucleotide headpiece is headpiece 1 (SEQ ID NO: 3), which has the following structure:

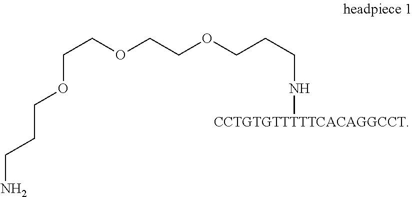

An example of a linker including a DBCO group is linker 2, which has the following structure:

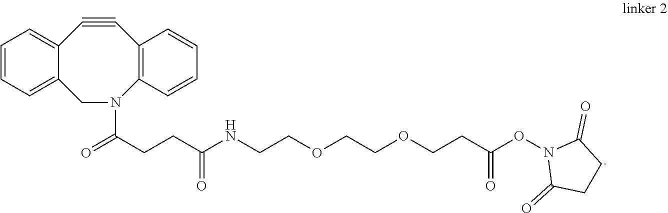 [SMILES: O=C(CCC(=O)N1Cc2ccccc2C#Cc2ccccc21)NCCOCCOCCC(=O)ON1C(=O)CCC1=O]

Headpiece 1 and linker 2 are reacted together so that the NHS ester group of linker 2 reacts with the amine group of headpiece 1 to generate a conjugate (conjugate 2; SEQ ID NO: 4), which includes an oligonucleotide headpiece and a DBCO cross-linking group:

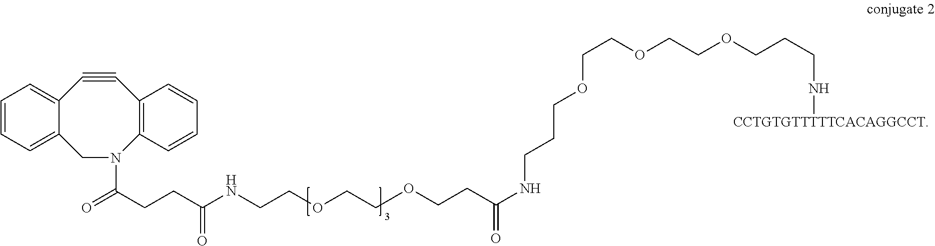

Conjugate 2 is purified using HPLC.

Reacting the First Conjugate with a Second Conjugate to Produce a Third Conjugate

To each well is then added conjugate 2 in aqueous buffer, and the resulting mixture is incubated to allow reaction (e.g., click chemistry) between the azide of conjugate 1 and the strained alkyne of conjugate 2 to produce conjugate 3 (SEQ ID NO: 5), which has the following structure:

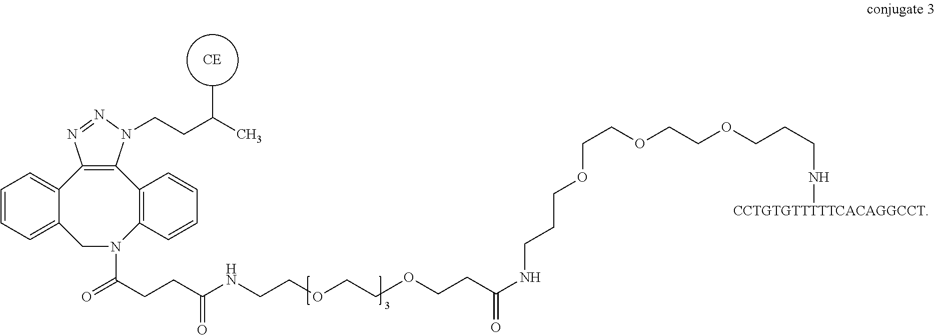

A simplified illustration of conjugate 3, as used herein, is shown below:

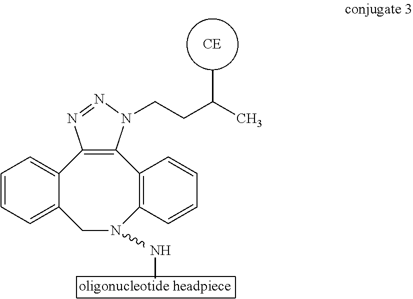

Ligating Oligonucleotide Tags to the Oligonucleotide Headpiece of the Third Conjugate

Subsequently an aqueous solution of a well-specific, and therefore compound-specific, collection of DNA tags is added that are designed to permit only one order of ligation by careful design of orthogonal overlap architectures.

An example of an encoding strategy is illustrated below:

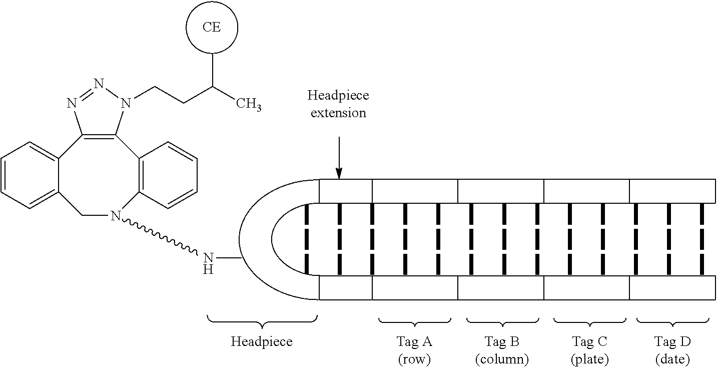 [SMILES: CNN1Cc2ccccc2-c2nnn(CCC(C)C)c2-c2ccccc21]

In this example compound collections are encoded using a four-register tag system in which compounds (e.g., pre-existing compounds) are presented in plates and Tag A encodes the identity of the row of each plate, Tag B encodes the identity of the column of each plate, Tag C encodes the identity of each plate, and variation at Tag D allows for the preceding tags to be subsequently reused in a different context. If 400 tags are available in total, divided equally between each register, then a total of 100 million compounds may each be uniquely encoded.

After the ligation incubation is complete then the contents of each well are combined and quenched, e.g., with EDTA, and the individual encoded compounds that comprise the entire library are thereby pooled together. The library is concentrated by precipitation and purified by HPLC. The library is then closed by a further ligation of a closing tag or tailpiece that introduces a library identifying sequence and a constant sequence for primer-binding during amplification and may optionally contain other tags and/or sequences helpful for downstream operations including clustering and sequencing.

Screening of Encoded Libraries

This library is then used to discover individual members that are able to bind to protein or other targets of interest by incubating with the target of interest, capture of the target, washing away of non-binding library members and the elution of the protein-associated members either by protein denaturation, tag cleavage or specific elution. The encoding DNA of the output population is then amplified and sequenced and compared with a corresponding sample derived from the input population to identify compounds that are enriched in the output. Compounds of interest are then sourced from the pre-existing collection and tested in target modulation assays to determine which may be considered hits.

### Example 2—Synthesis of a Conjugate DBCO-HP006 that Includes an Oligonucleotide Headpiece and a Cross-Linking Group

Headpiece HP006, chemically phosphorylated at its 5′ end, whose sequence is (p)CCTGTGTTZTTCACAGGCCT (SEQ ID NO: 6), where Z stands for the mdC(TEG-Amino) modification, was utilized.

To 300 μL of a solution of HP006 (10 mM) in water was added 8 μL of water, 25 μL of Pierce 1 M Borate Buffer pH 8.5 (Thermo Fisher), and 167 uL of a solution of DBCO-PEG4-NHS ester (BroadPharm) (30 mM) in DMSO. The mixture was allowed to stand at room temperature for 2 days. To a 62-μL fraction of this mixture was added ethanol (560 μL), and the precipitate was collected after centrifugation. The precipitate was washed with 80% ethanol (650 μL). The washed precipitate was allowed to dry by exposure to air, and then reconstituted in water (125 μL). The concentration of the product DBCO-HP006 was determined on a nanodrop UV spectrophotometer to be 2.7 mM (90%).

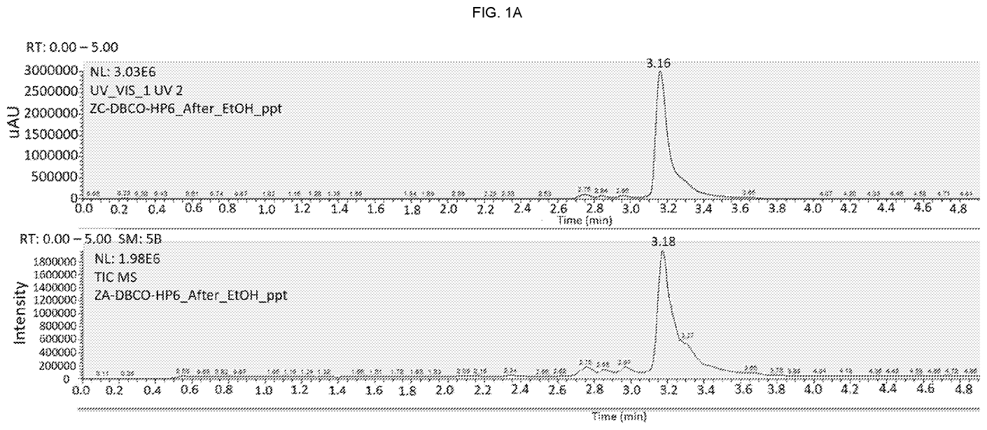

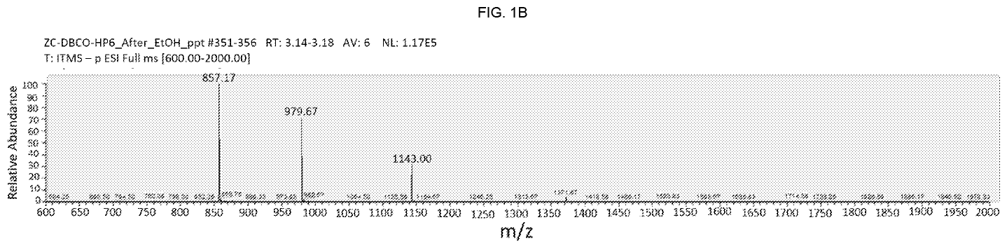

LCMS of the product DBCO-HP006 is shown in FIG. 1A and FIG. 1B. The mass spectrum confirmed the identity of the product (observed m/z in negative ion mode: 857.2, 979.7, 1143.0; calculated m/z: [M-8H]⁸⁻: 857.2, [M-7H]⁷⁻: 979.8, [M-6H]⁶⁻: 1143.2).

### Example 3—Conjugating Pre-Existing Compounds to Linker 1 and DBCO-HP006

To the bottom of each well of a 96-well natural-colored polypropylene PCR plate was added a DMSO solution containing 18 mM or 6 mM of a pre-existing compound and 200 mM of Linker 1. The plate was irradiated on an Alpha Innotech AIML-26 Transilluminator at 365 nm (6×8 W) for 10 minutes. A 2 μL fraction of each reaction mixture was then mixed into 20 μL of 25 μM DBCO-HP006 in 1×T4 DNA ligase buffer (made from 10× ligase buffer from Thermo Fisher) and allowed to stand at room temperature overnight.

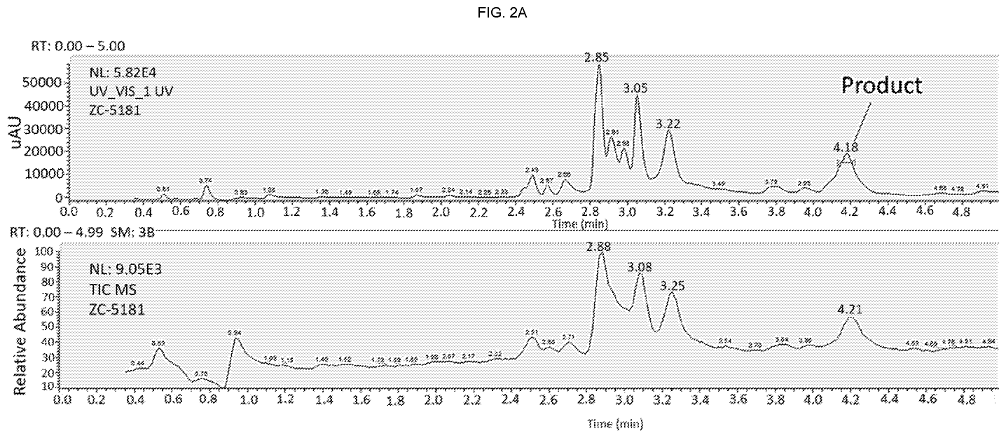

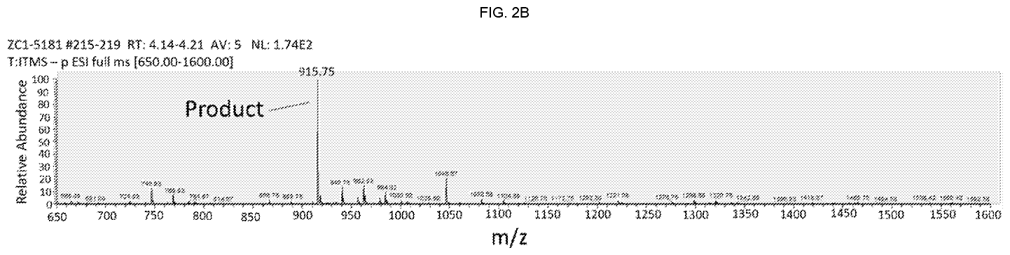

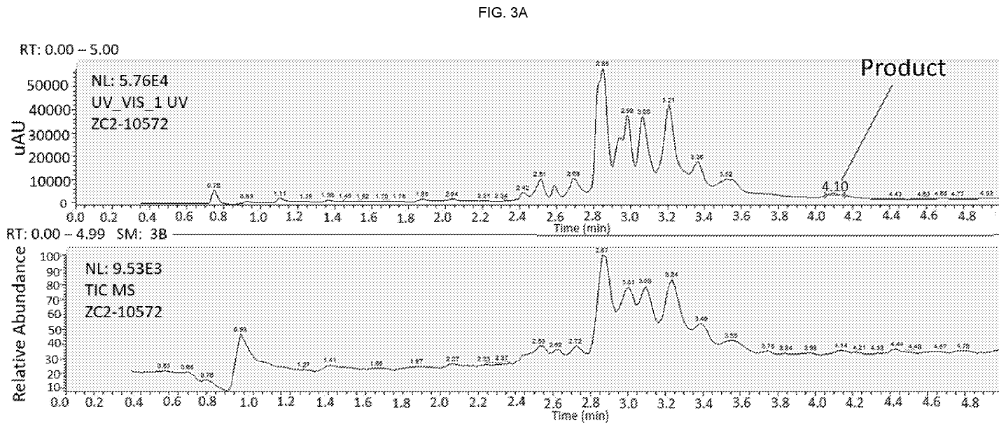

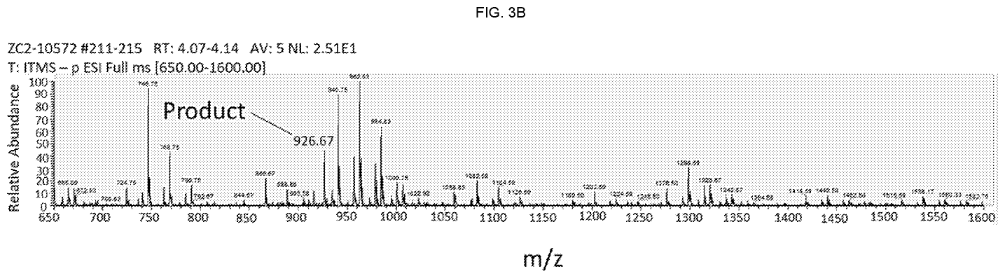

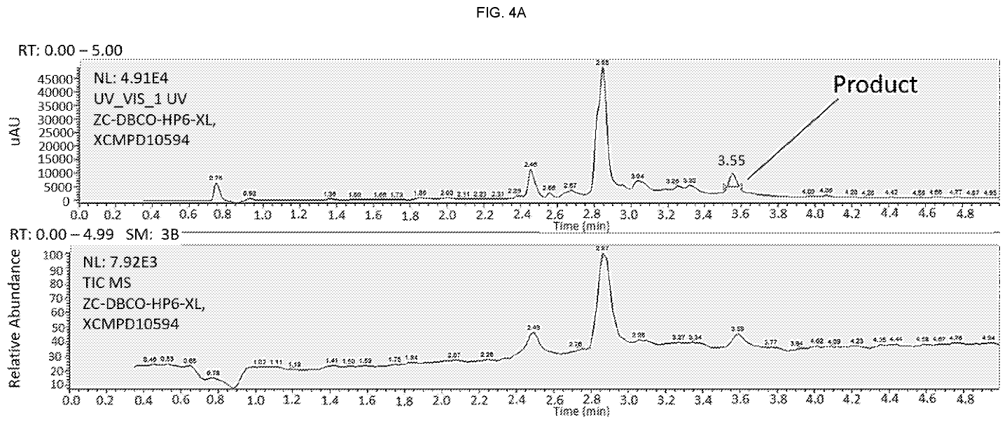

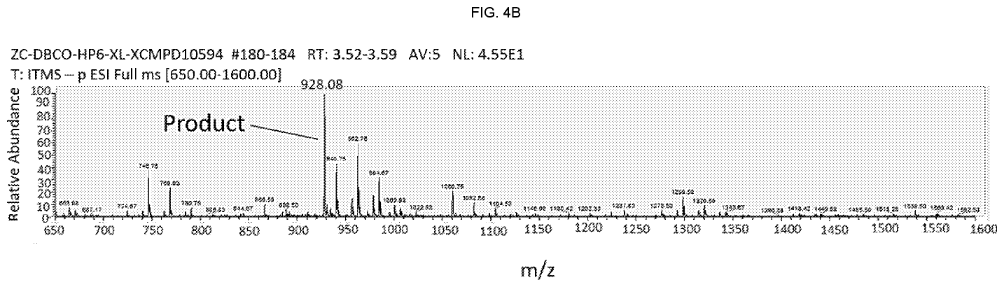

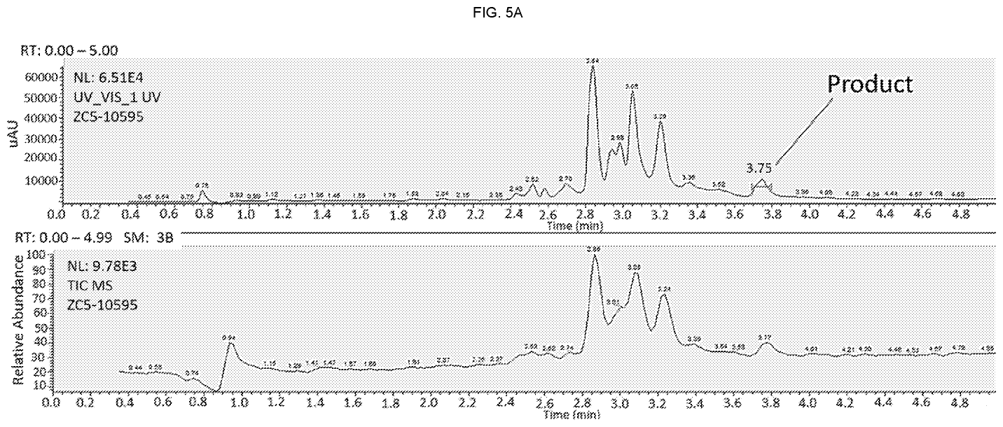

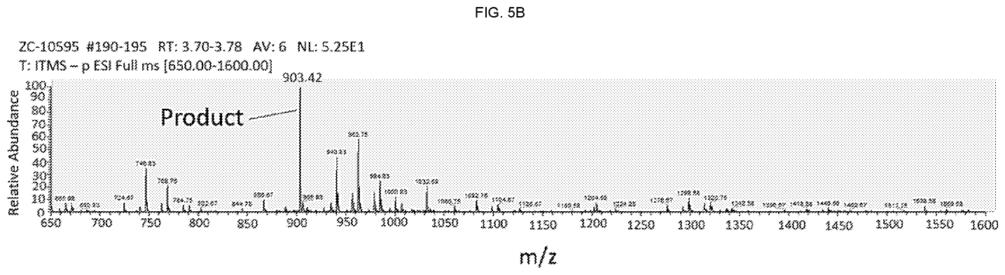

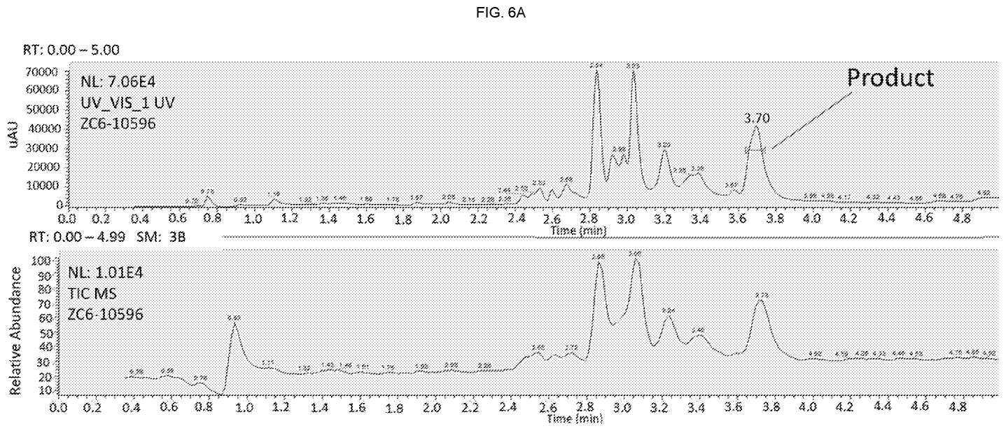

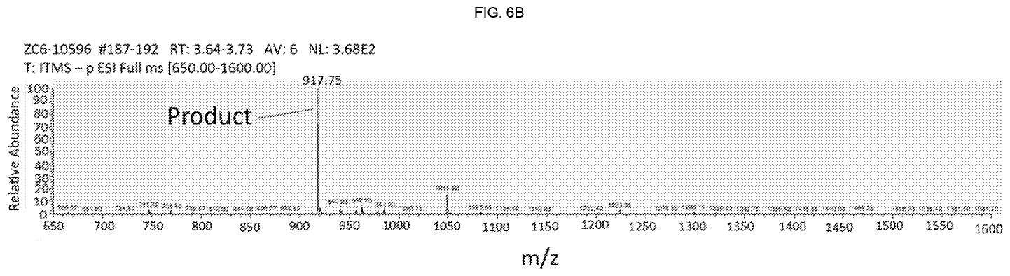

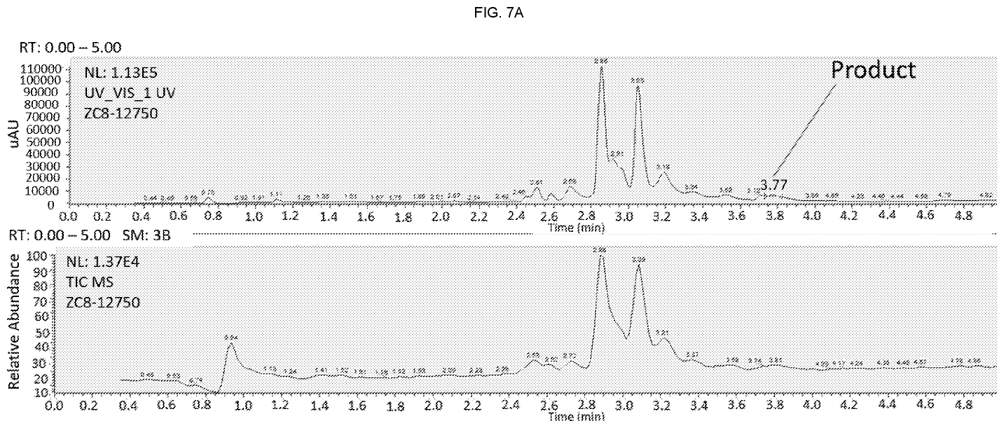

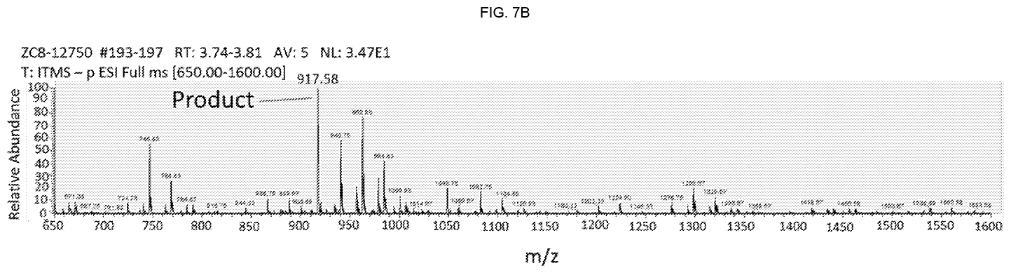

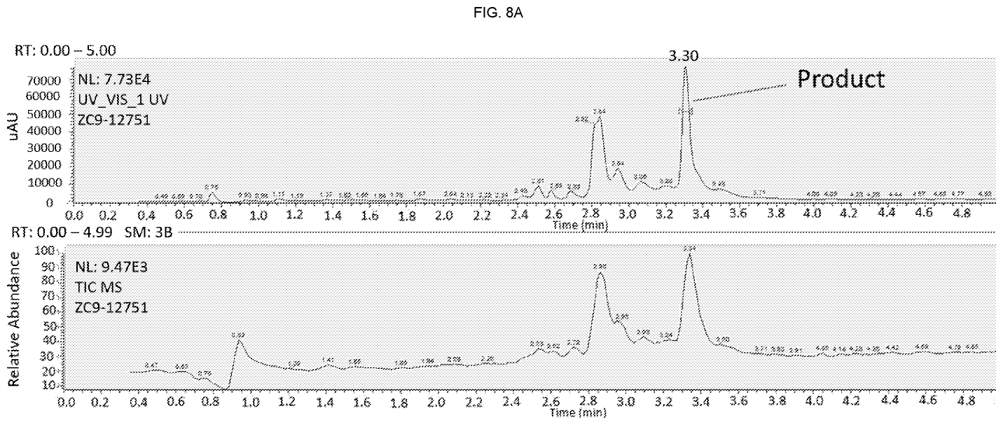

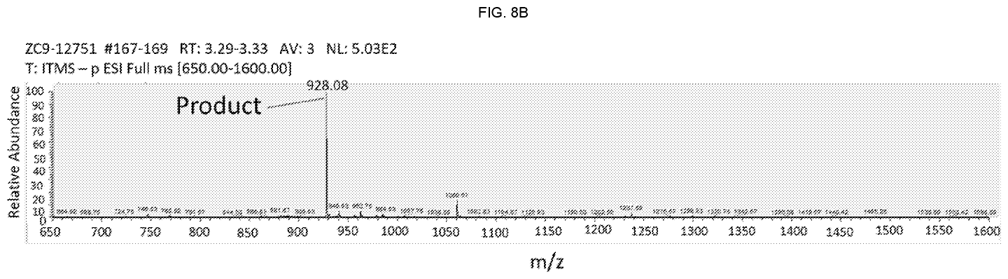

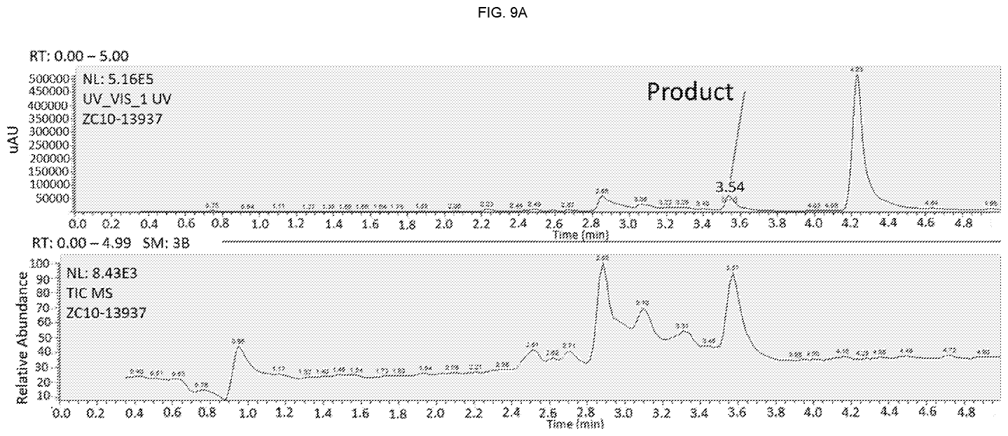

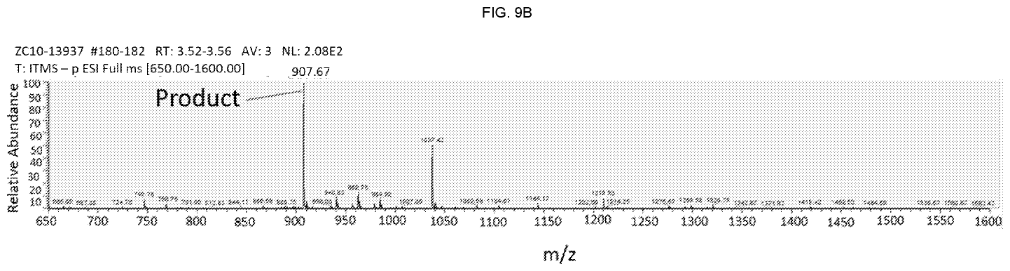

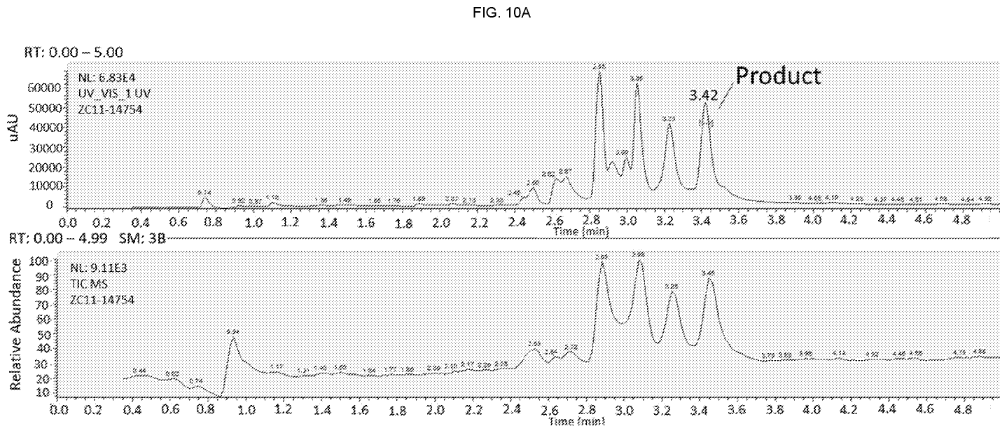

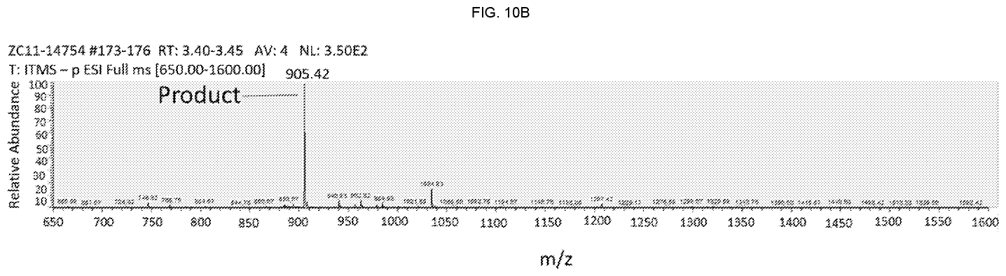

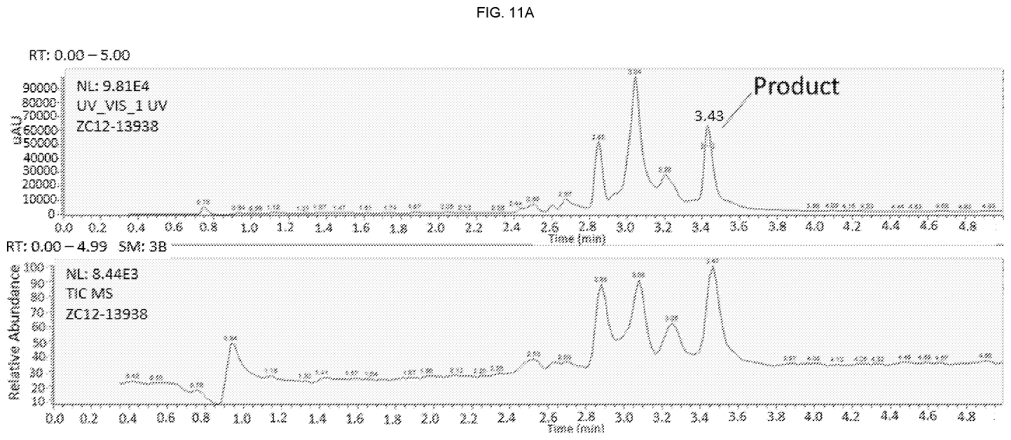

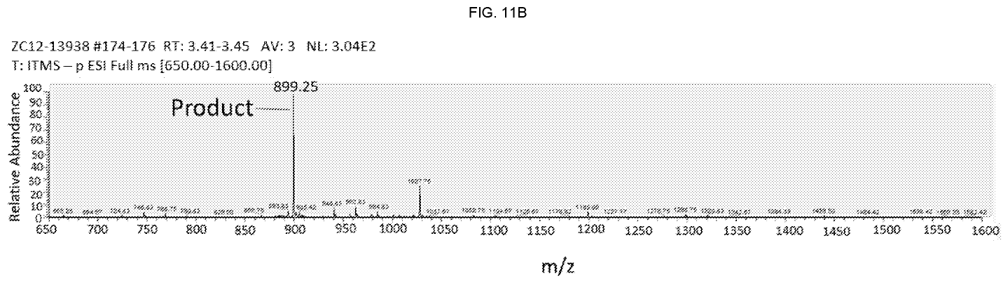

Twelve pre-existing compounds were subjected to this conjugation procedure (Table 1). The crude conjugation mixtures were analyzed by LCMS. Conjugation products with the expected m/z values could be detected for ten out of the twelve starting compounds. The results are summarized in Table 1. LCMS data are shown in FIG. 2A, FIG. 2B, FIG. 3A, FIG. 3B, FIG. 4A, FIG. 4B, FIG. 5A, FIG. 5B, FIG. 6A, FIG. 6B, FIG. 7A, FIG. 7B, FIG. 8A, FIG. 8B, FIG. 9A, FIG. 9B, FIG. 10A, FIG. 10B, FIG. 11A, and FIG. 11B.

TABLE 1
Summary of LCMS analysis results for conjugating
pre-existing compounds to Linker 1 and DBCO-HP006

| | Calculated m/z | |
|---|---|---|
| | for [M − 8H]⁸⁻ | |
| | of compound/ | |
| | linker 1/DBCO- | |
| Pre-existing compound | HP006 conjugate | Observed m/z |
| Tamoxifen | 915.9 | 915.8 |
| (CAS# 10540-29-1) | | (FIG. 2A and FIG. 2B) |
| Elacestrant, RAD1901 | 926.8 | 926.7 |
| (CAS# 722533-56-4) | | (FIG. 3A and FIG. 3B) |
| AZD 9496 | 924.8 | Expected product was |
| (CAS# 1639042-08-2) | | not observed. |
| Bazedoxifene | 928.3 | 928.1 |
| (CAS# 198481-32-2) | | (FIG. 4A and FIG. 4B) |
| 17β-Estradiol | 903.5 | 903.4 |
| (CAS# 50-28-2) | | (FIG. 5A and FIG. 5B) |
| (Z)-4-hydroxy Tamoxifen | 917.9 | 917.8 |
| (CAS# 68047-06-3) | | (FIG. 6A and FIG. 6B) |
| Fulvestrant | 945.3 | Expected product was |
| (CAS# 129453-61-8) | | not observed. |
| PPT | 917.8 | 917.6 |
| (CAS# 263717-53-9) | | (FIG. 7A and FIG. 7B) |
| MPP | 928.2 | 928.1 |
| (CAS# 911295-24-4) | | (FIG. 8A and FIG. 8B) |
| WAY 200070 | 907.8 | 907.7 |
| (CAS# 440122-66-7) | | (FIG. 9A and FIG. 9B) |
| Estriol | 905.5 | 905.4 |
| (CAS# 50-27-1) | | (FIG. 10A and FIG. 10B) |
| DPN | 899.4 | 899.3 |
| (CAS# 1428-67-7) | | (FIG. 11A and FIG. 11B) |

### Example 4—One-Pot Ligation of an Oligonucleotide Headpiece, a Headpiece Extension, and Four Tags that May be Used to Encode Compound Identity

Two DNA oligonucleotides, chemically phosphorylated at their respective 5′ ends, whose sequences are (p)TGGCTATCCTGGCTGAGG (SEQ ID NO: 7) and (p)CAGCCAGGATAG (SEQ ID NO: 8), were combined in equimolar ratio to make a 1 mM solution of double-stranded EXT00001.

Two DNA oligonucleotides, chemically phosphorylated at their respective 5′ ends, whose sequences are (p)CCAAAGAGTGGAGCTAAG (SEQ ID NO: 9) and (p)AGCTCCACTCTT (SEQ ID NO: 10), were used. as a pre-mixed 1 mM solution of double-stranded TagA.

Two DNA oligonucleotides, chemically phosphorylated at their respective 5′ ends, whose sequences are (p)GCTATGGAGCCACTACTT (SEQ ID NO: 11) and (p)TAGTGGCTCCAT (SEQ ID NO: 12), were used as a pre-mixed 1 mM solution of double-stranded TagB.

Two DNA oligonucleotides, chemically phosphorylated at their respective 5′ ends, whose sequences are (p)AGCGGATCTAGCCAATGC (SEQ ID NO: 13) and (p)TTGGCTAGATCC (SEQ ID NO: 14), were used as a pre-mixed 1 mM solution of double-stranded TagC.

Two DNA oligonucleotides, chemically phosphorylated at their respective 5′ ends, whose sequences are (p)CATACATACGCGACTGCA (SEQ ID NO: 15) and (p)AGTCGCGTATGT (SEQ ID NO: 16), were used as a pre-mixed 1 mM solution of double-stranded TagD.

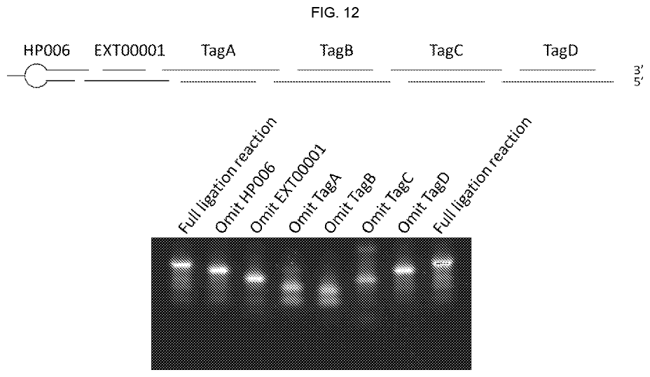

To a 50 μL mixture of six duplexed oligonucleotide components (final concentrations: 20 μM HP006, 1.05 molar equivalent of EXT00001, 1.1 molar equivalent of TagA, 1.15 molar equivalent of TagB, 1.2 molar equivalent of TagC, 1.25 molar equivalent of TagD) in 1× ligase buffer (using 10× ligase buffer from Thermo Fisher) was added 1.5 ul of T4 DNA ligase (Thermo Fisher). Six negative control reactions were set up with the same procedure except that, in each negative control reaction, one of the duplexed oligonucleotide components was replaced by an equivalent volume of water. The reactions were incubated at 16° C. in a thermocycler for 2 days. The reaction mixtures were analyzed by electrophoresis on a 4% E-Gel high-resolution agarose gel containing ethidium bromide. The gel image is shown in FIG. 12.

The one-pot ligation reaction produced one major DNA ligation product that is longer than the major products produced by all the negative control reactions, proving that the one-pot ligation reaction ligated all the oligonucleotide components in a defined sequence.

### OTHER EMBODIMENTS

All publications, patent applications, and patents mentioned in this specification are herein incorporated by reference.

Various modifications and variations of the described method and system will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

What is claimed is:

1. A method of producing an encoded chemical entity, the method comprising:
(a) reacting a chemical entity with a bifunctional linker, the bifunctional linker comprising a carbene precursor group and a first cross-linking group, under conditions sufficient to produce a first conjugate comprising the chemical entity and the first cross-linking group;
(b) reacting the first conjugate with a second conjugate, the second conjugate comprising an oligonucleotide headpiece and a second cross-linking group, under conditions sufficient to produce a third conjugate comprising the chemical entity and the oligonucleotide headpiece; and
(c) ligating a first oligonucleotide tag to the oligonucleotide headpiece of the third conjugate,
thereby producing an encoded chemical entity.

2. The method of claim 1, wherein the bifunctional linker is volatile.

3. The method of claim 1 or 2, wherein the bifunctional linker has the structure:
A-L¹-R¹ Formula I
wherein A is the carbene precursor group;
L¹ is a linker; and
R¹ is the first cross-linking group.

4. The method of any one of claims 1 to 3, wherein the carbene precursor group is a photo-reactive carbene precursor group.

5. The method of claim 4, wherein the photo-reactive carbene precursor group is a diazirine.

6. The method of any one of claims 1 to 5, wherein the carbene precursor group comprises the structure:

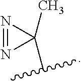 [SMILES: *C1(C)N=N1]

7. The method of any one of claims 3 to 6, wherein L¹ is C₁-C₆ alkylene.

8. The method of claim 7, wherein L¹ is C₂ alkylene.

9. The method of any one of claims 1 to 8, wherein the first cross-linking group is a sulfhydryl-reactive cross-linking group, an amino-reactive cross-linking group, a carboxyl-reactive cross-linking group, a carbonyl-reactive cross-linking group, or a triazole-forming cross-linking group.

10. The method of claim 9, wherein the first cross-linking group is a triazole-forming cross-linking group.

11. The method of any one of claims 1 to 10, wherein the first cross-linking group is an azide.

12. The method of any one of claims 1 to 11, wherein the bifunctional linker has the structure:

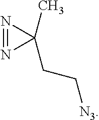

13. The method of any one of claims 1 to 12, wherein the second conjugate has the structure:
B-L²-R² Formula II
wherein B is the oligonucleotide headpiece;
L² is a linker; and
R² is the second cross-linking group.

14. The method of any one of claims 1 to 13, wherein the oligonucleotide headpiece comprises a hairpin structure.

15. The method of claim 13 or 14, wherein the second cross-linking group is a sulfhydryl-reactive cross-linking group, an amino-reactive cross-linking group, a carboxyl-reactive cross-linking group, a carbonyl-reactive cross-linking group, or a triazole-forming cross-linking group.

16. The method of claim 15, wherein the second cross-linking group is a triazole-forming cross-linking group.

17. The method of claim 16, wherein the second cross-linking group comprises a dibenzocyclooctyne group.

18. The method of claim 17, wherein the second cross-linking group comprises the structure:

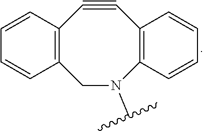 [SMILES: *N1Cc2ccccc2C#Cc2ccccc21]

19. The method of any one of claims 1 to 18, wherein the method further comprises producing the second conjugate by reacting a fourth conjugate comprising an oligonucleotide headpiece and a cross-linking group with a fifth conjugate of Formula III:
R³-L³-R⁴ Formula III
wherein R³ and R⁴ are, independently, cross-linking groups; and
L³ is a linker,
under conditions sufficient to produce the second conjugate.

20. The method of claim 19, wherein R³ is a triazole-forming cross-linking group.

21. The method of claim 20, wherein R³ comprises a dibenzocyclooctyne group.

22. The method of claim 20, wherein R³ comprises the structure:

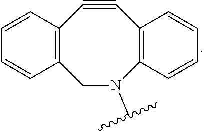 [SMILES: *N1Cc2ccccc2C#Cc2ccccc21]

23. The method of any one of claims 19 to 22, wherein R⁴ is a sulfhydryl-reactive cross-linking group, an amino-reactive cross-linking group, a carboxyl-reactive cross-linking group, a carbonyl-reactive cross-linking group, or a triazole-forming cross-linking group.

24. The method of claim 23, wherein R⁴ is an amino-reactive cross-linking group.

25. The method of claim 24, wherein R⁴ comprises a N-hydroxysuccinimide group.

26. The method of any one of claims 19 to 25, wherein the second conjugate has the structure:
B-L⁴-R⁵ Formula IV
wherein B is the oligonucleotide headpiece;
L⁴ is a linker; and
R⁵ is the second cross-linking group.

27. The method of claim 26, wherein the second cross-linking group is an amino group.

28. The method of any one of claims 1 to 27, wherein the method further comprises, prior to step (c), ligating a headpiece extension sequence to the headpiece.

29. The method of any one of claims 1 to 28, wherein the method further comprises ligating one or more further tags to the encoded chemical entity after step (c).

30. The method of claim 29, wherein the method further comprises ligating at least three further tags to the encoded chemical entity after step (c).

31. The method of claim 30, wherein the method comprises one-pot ligation.

32. The method of claim 31, wherein the one-pot ligation comprises the ligation of the headpiece extension sequence to the headpiece and the ligation of the at least three further tags to the encoded chemical entity.

33. The method of any one of claims 29 to 32, wherein the first oligonucleotide tag and the one or more further tags comprise orthogonal overlap architectures.

34. The method of any one of claims 1 to 33, wherein the method further comprises ligating a tailpiece to the encoded chemical entity.

35. The method of any one of claims 1 to 34, wherein the chemical entity does not comprise an N—H or O—H bond.

36. The method of any one of claims 1 to 35, wherein the conditions of step (b) do not comprise a metal catalyst.

37. The method of any one of claims 1 to 36, wherein the method further comprises purifying the encoded chemical entity after step (c).

38. The method of claim 37, wherein the purifying comprises high performance liquid chromatography (HPLC).

39. The method of any one of claims 1 to 38, wherein the conditions of step (a) comprises irradiation.

40. A library comprising a plurality of encoded chemical entities produced by the method of any one of claims 1 to 39.

41. The library of claim 40, wherein the plurality of encoded chemical entities is not physically separated.

42. The library of claim 40 or 41, wherein the plurality of encoded chemical entities comprises at least 1,000,000 different chemical entities.

43. The library of any one of claims 40 to 42, wherein the plurality of encoded chemical entities comprises at least 5,000,000 different chemical entities.

44. The library of any one of claims 40 to 43, wherein the plurality of encoded chemical entities comprises at least 10,000,000 different chemical entities.

45. The library of claim 40 or 41, wherein the plurality of encoded chemical entities comprises about 1,000,000 to about 5,000,000 different chemical entities.

46. The library of claim 40 or 41, wherein the plurality of encoded chemical entities comprises about 5,000,000 to about 10,000,000 different chemical entities.

47. A method of screening a plurality of chemical entities, the method comprising:
(a) contacting a target with an encoded chemical entity prepared by a method of any one of claims 1 to 39 and/or a library of any one of claims 40 to 46; and
(b) selecting one or more encoded chemical entities having a predetermined characteristic for the target, as compared to a control, thereby screening a plurality of the chemical entities.

48. The method of claim 47, where the predetermined characteristic comprises increased binding for the target, as compared to a control.


SEQUENCE LISTING (16 sequences)

SEQ ID NO: 1 - DNA, 7 bp, Artificial Sequence
  Synthetic Construct
gtgctgc

SEQ ID NO: 2 - DNA, 9 bp, Artificial Sequence
  Synthetic Construct
gcagcaccc

SEQ ID NO: 3 - DNA, 20 bp, Artificial Sequence
  Synthetic Construct
  misc_feature (9)..(9): N is Thymine modified with TEG-amino
cctgtgttnttcacaggcct

SEQ ID NO: 4 - DNA, 20 bp, Artificial Sequence
  Synthetic Construct
  misc_feature (9)..(9): N is Thymine modified with TEG-amino and dibenzocyclooctyne-amino
cctgtgttnttcacaggcct

SEQ ID NO: 5 - DNA, 20 bp, Artificial Sequence
  Synthetic Construct
  misc_feature (9)..(9): N is Thymine modified with TEG-amino and triazole-amino
cctgtgttnttcacaggcct

SEQ ID NO: 6 - DNA, 20 bp, Artificial Sequence
  Synthetic Construct
  misc_feature (9)..(9): N is Cytosine modified with TEG-amino
cctgtgttnttcacaggcct

SEQ ID NO: 7 - DNA, 18 bp, Artificial Sequence
  Synthetic Construct
tggctatcctggctgagg

SEQ ID NO: 8 - DNA, 12 bp, Artificial Sequence
  Synthetic Construct
cagccaggatag

SEQ ID NO: 9 - DNA, 18 bp, Artificial Sequence
  Synthetic Construct
ccaaagagtggagctaag

SEQ ID NO: 10 - DNA, 12 bp, Artificial Sequence
  Synthetic Construct
agctccactctt

SEQ ID NO: 11 - DNA, 18 bp, Artificial Sequence
  Synthetic Construct
gctatggagccactactt

SEQ ID NO: 12 - DNA, 12 bp, Artificial Sequence
  Synthetic Construct
tagtggctccat

SEQ ID NO: 13 - DNA, 18 bp, Artificial Sequence
  Synthetic Construct
agcggatctagccaatgc

SEQ ID NO: 14 - DNA, 12 bp, Artificial Sequence
  Synthetic Construct
ttggctagatcc

SEQ ID NO: 15 - DNA, 18 bp, Artificial Sequence
  Synthetic Construct
catacatacgcgactgca

SEQ ID NO: 16 - DNA, 12 bp, Artificial Sequence
  Synthetic Construct
agtcgcgtatgt